(19) United States
(12) Patent Application Publication
(10) Pub. No.: US 2025/0340938 A1
(43) Pub. Date: Nov. 6, 2025

(54) MULTIPLEX PCR REACTION SYSTEM

(71) Applicant: MACCURA BIOTECHNOLOGY CO., LTD., Chengdu, Sichuan (CN)

(72) Inventors: Yuhang ZHAO, Chengdu, Sichuan (CN); Wan'er HU, Chengdu, Sichuan (CN); Haomiao ZHAN, Chengdu, Sichuan (CN)

(73) Assignee: MACCURA BIOTECHNOLOGY CO., LTD., Chengdu, Sichuan (CN)

(21) Appl. No.: 18/724,419

(22) PCT Filed: Dec. 27, 2022
(86) PCT No.: PCT/CN2022/142537

§ 371 Date: Jun. 26, 2024

(30) Foreign Application Priority Data
Dec. 27, 2021 (CN) 202111620423.X

(51) Int. Cl. C12Q 1/6876; C12Q 1/686

(52) U.S. Cl. CPC C12Q 1/6876; C12Q 1/686; C12Q 2600/16

(57) ABSTRACT

Provided in the present invention is a method for detecting and differentiating multiple different targets in a single-tube PCR reaction by using single-tube ultra-multiplex PCR. The method can avoid false positive signals generated by a primer dimer when multiple specific primers are present. Further provided in the present invention are a primer and probe for the single-tube super-multiplex PCR, a reaction system, and a multiple-target detection kit containing the primer, the probe and the reaction system.

The entire contents of the electronic file identified by its name WO11605MK-2.xml, creation date Dec. 27, 2024, and size 45,085 bytes are incorporated herein by reference. Neither the presence nor absence of information that is not required under the sequence rules will create a presumption that such information is necessary to satisfy any of the requirements of 35 U.S.C. 112. Further, the grant of a patent on an application that is subject to the sequence rules (37 CFR 1.831 et seq.) constitutes a presumption that the granted patent complies with the requirements of these rules.

### FIELD OF THE INVENTION

The present invention belongs to the field of molecular biology, in particular to the field of nucleic acid amplification and detection.

### BACKGROUND OF THE INVENTION

Polymerase chain reaction (PCR) is a molecular biological technology that performs DNA replication via enzyme without using living organisms. The PCR is usually used in medical and biological research laboratories to undertake a variety of tasks, such as diagnosis of infectious diseases, gene cloning, phenotypic identification of laboratory animals, research on transcriptomes, detection of genetic diseases, identification of gene fingerprints, paternity test, etc. Since the PCR has an incomparable replication ability and accuracy, it is considered by the molecular biologists as a preferred method for nucleic acid detection. In the late 1990s, a real-time fluorescence quantitative PCR (qPCR) technology and related products launched by ABI in the USA further developed PCR to be a highly sensitive, highly specific and accurately quantitative nucleic acid sequence analysis technology.

At present, a primer probe design method that is widely used in qPCR platforms is mainly a TaqMan hydrolysis probe method, a working principle of which is mainly characterized by using an oligonucleotide probe that can specifically bind with a template and is labeled with a fluorescence group (donor) and a quencher group (receptor) at two ends thereof, and by designing a specific PCR primer upstream and downstream of the probe respectively. Before the start of PCR reaction, due to a principle of fluorescence resonance energy transfer (FRET), a fluorescence signal emitted by the fluorescence group at one end of the TaqMan probe is absorbed by the quencher group at the other end, such that the fluorescence signal cannot be detected by an instrument; but after the start of PCR amplification, the TaqMan probe specifically binds with the template, and when a DNA polymerase (Taqase) extends to a site at which the probe binds to the template, the TaqMan probe will be cleaved due to 5′-3′ exonuclease activity of the DNA polymerase (Taqase), such that the fluorescence group labeled on the probe will be far away from the quencher group and a FRET structure cannot be formed any more, and thus a signal emitted by the fluorescence group can be detected by the instrument. However, if a PCR detection for multiplex targets is intended to be achieved in a same reaction system, it is required to arrange multiple TaqMan hydrolysis probes labeled with fluorescence groups of different wavelengths, and at present, only a detection of 4-6-plex different targets can be achieved at most due to limitation of a number of fluorescent channels in a detection instrument.

In order to achieve an ultra-multiplex PCR detection, in the prior art, the PCR detection technology is usually combined with other methods, for example, the PCR is combined with a hybrid chip to perform detection of multiplex targets with different amplification product lengths. For example, a patent document CN107090519A discloses a detection kit for common respiratory pathogens combining multiplex RT-PCR with a gene chip, in which, a principle of nucleic acid molecule hybridization is utilized, single-stranded probes targeting individual targets are arranged and fixed on the gene chip in a specific order to form a probe array, and then multiplex PCR products to be detected are allowed to be hybridized with it, such that target amplification products are hybridized with the probes on the chip and emit fluorescence signals. Although 20 types of respiratory pathogens can be simultaneously detected by this method, the gene chip is complex in preparation process, and is expensive, thereby being not conducive to clinical promotion. Moreover, the method is cumbersome in procedures, requires multiple steps of uncovering and cleaning, and may easily cause contamination and result in a false positive result.

For another example, a patent document CN103074450B discloses a kit for simultaneous detection of thirty types of pathogenic bacteria causing diarrhea and a detection method thereof. The method combines the PCR amplification and capillary electrophoresis. In the method, PCR amplification products are taken out and subjected to capillary electrophoresis analysis; and the obtained spectrum is compared with a standard spectrum to determine the types of pathogenic bacteria causing diarrhea. In addition to the use of a PCR amplification detection equipment, this method further requires the use of a capillary electrophoresis equipment for product analysis, such that a detection cost is greatly increased, thereby being not conducive to clinical promotion and application either.

Therefore, there has been an urgent clinical need for a method for multiplex nucleic acid molecule detection, which is fast, simple, and low-cost for pathogenic microorganism detection, non-invasive birth defect screening, single nucleotide polymorphism analysis, methylation analysis, gene mutation analysis, or gene typing, etc.

### SUMMARY OF THE INVENTION

The problem to be solved by the present invention is to detect and differentiate multiple different targets in a single-tube PCR reaction by using single-tube ultra-multiplex PCR. The method can avoid false positive signals generated by a primer dimer when multiple specific primers exist. The present invention further provides a primer and probe for the single-tube super-multiplex PCR, a reaction system, and a multiple-target detection kit containing the primer, the probe and the reaction system.

In a first aspect, the present invention provides a probe primer composition for PCR detection, comprising a probe and a first primer set, wherein the first primer set comprises a first primer and a second primer that are a forward primer and a reverse primer or a reverse primer and a forward primer, respectively, wherein
the probe comprises a first binding region A′ and a second binding region H,
the first primer comprises a sequence region A that is partially or completely complementary with the first binding region A′ of the probe, and the second primer comprises a sequence region h that is partially or completely identical to the second binding region H of the probe,
none of the first binding region A′, the second binding region H, the sequence region A and the sequence region h are complementary or identical to a target sequence or any fragment thereof, and
the probe has a detectable label thereon.

In some embodiments, the first primer is a forward primer, and the second primer is a reverse primer; or the first primer is a reverse primer, and the second primer is a forward primer.

In some embodiments, one single-stranded amplification product of the double-stranded amplification product of the first primer and the second primer comprises the sequence region A and a sequence h′ that is partially or completely complementary with the second binding region H of the probe.

In some embodiments, the probe has a length of 20-100 bases.

In some embodiments, the first binding region A′ of the probe has a length of 5-65 bases.

In some embodiments, the first binding region A′ of the probe has a length of 5-35 bases.

Preferably, the first binding region A′ has a Tm value of 30° C.-80° C., and preferably 40° C.-80° C.

Preferably, the first binding region A′ has a GC content of 30%-80%.

Preferably, the first binding region A′ is designed at a 5′ or 3′ end of the probe, and is completely or partially complementary with the sequence region A of the first primer.

Preferably, a blocking region is provided at the 3′ end of the probe.

The blocking region may prevent the probe from extending at the 3′ end.

The blocking region is a portion that is configured to prevent extension of the nucleic acid strand through DNA polymerase, thereby blocking strand extension during, for example, a PCR process. The blocking region may function as follows: 3′OH is labeled by 3′-Spacer C3, 3′-Phosphat, 3′-ddC, 3′-InvertedEnd, etc., such that the 3′ OH is blocked, so as to prevent its extension reaction. The blocking region may also be a polymerase enzyme blocking group, which is a group that has a property function of blocking further elongation of a polymer. The blocking group may be any chemical group that can connect to a nucleotide, which allows a 5′ end of a labeled nucleotide to connect to a 3′ end of another nucleotide in a DNA strand but does not allow a nucleotide to connect to a 3′hydroxyl group of the labeled nucleotide. Properly, the absence of OH group at 3′ position will prevent further extension by means of polymerase activity. In some embodiments, the blocking group is selected from acetyl, CH3, glycyl, lencyl, and alanyl groups. In some other embodiments, the blocking group may be in a form of a dipeptide or tripeptide.

In some embodiments, the second binding region H has a length of 5-65 bases.

Preferably, the second binding region H has a Tm value of 30° C.-80° C.

Preferably, the second binding region H has a GC content of 20%-80%.

In some embodiments, the detectable label comprises a first detectable label and a second detectable label, wherein the first detectable label is a fluorescence reporter, and the second detectable label is a quencher or other labeling groups that can produce a signal change with the first detectable label through fluorescence resonance energy transfer.

In some embodiments, the second detectable label is spaced apart from the first detectable label by 3-250 angstroms; preferably, the second detectable label is spaced apart from the first detectable label by 3-201 angstroms; and more preferably, the second detectable label is spaced apart from the first detectable label by 3-140 angstroms.

Preferably, the second detectable label is at a position spaced apart from the first detectable label by 5 to 25 bases.

In some embodiments, both of the first detectable label and the second detectable label are located on the first binding region A′, or both of the first detectable label and the second detectable label are located on the second binding region H.

In some embodiments, at least one of the first detectable label and the second detectable label is located on the first binding region A′, or at least one of the first detectable label and the second detectable label is located on the second binding region H.

In some embodiments, a spacer region between the first detectable label and the second detectable label is at least partially located on the first binding region A′, or the spacer region between the first detectable label and the second detectable label is at least partially located on the second binding region H.

In some embodiments, the first primer further comprises a first target sequence binding region, and the second primer further comprises a second target sequence binding region.

Preferably, the first primer sequentially comprises the sequence region A and the first target sequence binding region from 5′ end to 3′ end.

Preferably, the target sequence binding region is a target specific sequence that specifically binds with a target sequence to be amplified.

In some embodiments, an annealing temperature between the probe and the first primer is TmA, an annealing temperature between the probe and a complementary sequence of the second primer is TmH, an annealing temperature between the first primer and the target sequence is Tmb1, and an annealing temperature between the second primer and the target sequence is Tmb2. In some specific embodiments, TmA<Tmb1 and TmA<Tmb2, and/or TmH<Tmb1 and TmH<Tmb2.

In some embodiments, the first binding region A′ is complementary with the sequence region A of the first primer.

Preferably, the sequence region A of the first primer is a freely designed sequence that does not bind with the target sequence or any fragment thereof in a paired manner, and is partially or completely complementary with the first binding region A′ of the probe. More preferably, the sequence region A of the first primer is partially or completely complementary with the first binding region A′ of the probe.

Preferably, there are 0-20 bases spaced between the sequence region A and the target sequence binding region of the first primer.

Preferably, the first primer has a length of 20-80 bases.

Preferably, the first target sequence binding region of the first primer has a Tm value of 40° C.-80° C.

Preferably, the first target sequence binding region of the first primer has a GC content of 20%-80%.

Preferably, the sequence region A of the first primer has a length of 5-65 bases.

Preferably, the sequence region A of the first primer has a Tm value of 40° C.-80° C.

Preferably, the sequence region A of the first primer has a GC content of 30%-80%.

In some embodiments, the second binding region H and the sequence region h of the second primer have a partially or completely identical sequence.

In some embodiments, the second primer sequentially comprises the sequence region h and the second target sequence binding region from 5′ end to 3′ end.

Preferably, the target sequence binding region is a target specific sequence, and specifically binds with a target sequence to be amplified.

Preferably, the second primer further comprises an extension blocking region, which is a sequence that is not identical to or complementary with a base sequence of any part of the probe.

In some embodiments, the second primer sequentially comprises the extension blocking region, the sequence region h, and the second target sequence binding region from 5′ end to 3′ end.

Preferably, the sequence region h of the second primer is a freely designed sequence that does not bind with the target sequence in a paired manner, and has a sequence partially or completely identical to that of the second binding region H of the probe.

Preferably, there are 0-20 bases spaced between the sequence region h and the target sequence binding region.

Preferably, the second primer has a length of 20-80 bases.

Preferably, the extension blocking region of the second primer has a length of 1-20 bp.

Preferably, the second target sequence binding region of the second primer has a Tm value of 40° C.-80° C.

Preferably, the second target sequence binding region of the second primer has a GC content of 20%-80%.

Preferably, the sequence region h of the second primer has a length of 5-65 bases.

Preferably, the sequence region h of the second primer has a Tm value of 30° C.-80° C.

Preferably, the sequence region h of the second primer has a GC content of 20%-80%. In some embodiments, when there is no target to be amplified, the first binding region A′ of the probe completely or partially binds with the sequence region A of the first primer in a complementary manner, and the other part of the probe is in a single-stranded state. At this time, the single-stranded flexible probe is formed as a curly shape due to molecular flexibility, at this time, a distance between the first detectable label and the second detectable label is relatively close, and the efficiency of fluorescence resonance energy transfer is relatively high.

In some embodiments, during PCR amplification, the first primer and the second primer specifically bind with the target sequence respectively and extend to produce a double-stranded pre-amplification product. One of first single-stranded pre-amplification products of the double-stranded pre-amplification product comprises the sequence region A, the target sequence, and a complementary sequence h′ of the sequence region h; and the second binding region H of the probe is complementarily paired with the complementary sequence h′ of the sequence region h of the first single-stranded pre-amplification product, and the first binding region A′ of the probe is complementarily paired with the sequence region A of the first single-stranded pre-amplification product. The probe is changed from a single-stranded state to a double-stranded state, and a distance between the first detectable label and the second detectable label is increased, thereby generating a signal that may be detected by an instrument.

In some embodiments, both of the first detectable label and the second detectable label of the probe are located on the first binding region A′; or both of the first detectable label and the second detectable label of the probe are located on the second binding region H; or a spacer region between the first detectable label and the second detectable label is at least partially located on the first binding region A′; or a spacer region between the first detectable label and the second detectable label is at least partially located on the second binding region H. During PCR amplification, the first primer and the second primer specifically bind with the target sequence and extend to produce a double-stranded pre-amplification product. One of first single-stranded pre-amplification products of the double-stranded pre-amplification product comprises the sequence region A, the target sequence, and a complementary sequence h′ of the sequence region h; and the second binding region H of the probe is complementarily paired with the complementary sequence h′ of the sequence region h of the first single-stranded pre-amplification product, and the first binding region A′ of the probe is complementarily paired with the sequence region A of the first single-stranded pre-amplification product. The first single-stranded pre-amplification product forms an omega (a) hybridization form or an open-loop hybridization form with the probe, and the distance between the first detectable label and the second detectable label is increased, thereby generating a signal that may be detected by the instrument.

Preferably, the first single-stranded pre-amplification product further comprises a complementary sequence of an extension blocking region at 3′ end thereof, which is not complementary with any part of the probe.

In some embodiments, a target sequence amplification is identified by means of a fluorescence channel, or a target sequence amplification is identified by means of a melting temperature curve, or a target sequence amplification is identified by means of a fluorescence channel+a melting temperature curve.

Preferably, a target sequence amplification is identified by means of the melting temperature curve. Specifically, after PCR amplification, a melting curve analysis is performed, during a process of temperature change, the double-stranded product formed by the probe and the first single-stranded amplification product will melt at a certain temperature, at this time, the probe with the first detectable label and the second detectable label will be changed from a double strand to a single strand, such that the distance between the detectable labels is changed, thereby generating a signal that may be detected by the instrument.

In some embodiments, the probe primer composition for n target sequences comprises n first primer sets and m probes, wherein 1≤n≤20, and 1≤m≤n;

Base sequences of different probes are different, and the detectable labels on the probes are different.

Single-stranded amplification products obtained by amplifying different target sequences with all of the first primer sets for the same probe are different from each other in an annealing temperature with the probe.

Preferably, single-stranded amplification products obtained by amplifying different target sequences with all of the first primer sets for the same probe are different from each other by a ΔTm of 6-10° C. in an annealing temperature with the probe

Preferably, the target sequence binding regions included in all of the first primers are different from each other, and the target sequence binding regions included in all of the second primers are different from each other.

Preferably, the probe primer composition for n target sequences comprises n first primer sets and 1 or several probes, wherein 1≤n≤20.

In some embodiments, n probes are designed for n target sequences, and then an identification of amplifications of n target sequences is carried out by using a fluorescence channel. In such embodiments, the n probes are flexible probes.

The flexible probe forms a curly shape due to molecular flexibility in single-stranded state, but after it is extended to be double-stranded state by hybridization or annealing, it is fixed to form a relatively rigid double-helix structure due to hydrogen-bond interaction. The flexible probe may be selected from a group consisting of: an oligonucleotide that forms a curly shape due to molecular flexibility in single-stranded state, an oligonucleotide that can form a hairpin structure in single-stranded state, an oligonucleotide that can form a stem-loop structure in single-stranded state, an oligonucleotide that can form a pseudoknot structure in single-stranded state, and an oligonucleotide that can form a triplex structure in single-stranded state.

In some embodiments, only 1 or several commonly used probes are designed for n target sequences, and then amplifications of n target sequences are identified by using melting curves. In such embodiments, a sequence and/or length of the sequence region h of the second primer is adjusted, and/or a sequence and/or length of the sequence region A of the first primer is adjusted to obtain the sequence region A and the complementary sequence of the sequence region h of the first single-stranded pre-amplification product which are partially or completely complementary with the second binding region H of the probe, and partially or completely complementary with the first binding region A′, such that a melting temperature of the double-stranded product formed by the probe and the first single-stranded amplification product is adjusted.

Preferably, a length or sequence of the sequence region A and/or the sequence region h is adjusted, or positions at which the sequence region A and/or the complementary sequence of the sequence region h are complementary with the first binding region A′ and/or the second binding region H of the probe are adjusted, the melting temperature of the double-stranded product formed by the probe and the first single-stranded amplification product may be increased or decreased.

Such embodiments can achieve super-multiplex PCR in a single tube and effectively avoid the formation of primer dimers.

In some embodiments, there is provided a probe primer composition for detecting respiratory syncytial virus (RSV), comprising a probe, a forward primer, and a reverse primer selected from the following group:
a probe with a sequence shown in SEQ ID NO: 1;
a forward primer with a sequence shown in SEQ ID NO: 3; and
a reverse primer with a sequence shown in SEQ ID NO: 2.

In some embodiments, the reaction conditions are as follows: optionally, performing reverse transcription at 35-65° C. for 5-60 minutes; performing pre-denaturation at 90° C.-96° C. for 5-15 minutes; performing denaturation at 90° C.-95° C. for 10-60 seconds, and performing annealing and extension at 50° C.-75° C. for 30-90 seconds, for 35-50 cycles; optionally, performing denaturation at 90° C.-95° C. for 10-600 seconds, and performing incubation at a constant temperature of 30° C.-55° C. for 10-600 seconds; and performing melting curve analysis at 35-95° C. with a heating rate of 0.05° C./s, and collecting fluorescence signals.

When target sequence to be detected is RNA, a reverse transcription step may be used. When the target sequence to be detected is DNA, a reverse transcription step may not be used.

More preferably, the reaction conditions are as follows: performing reverse transcription at 55° C. for 15 minutes; performing pre-denaturation at 95° C. for 2 minutes; performing denaturation at 94° C. for 10 seconds, and performing annealing and extension at 56° C. for 30 seconds, for a total of 45 cycles; performing denaturation at 94° C. for 150 seconds, and performing incubation at a constant temperature of 40° C. for 300 seconds; and performing melting curve analysis at 40-95° C. with a heating rate of 0.05° C./S, and collecting fluorescence signals.

Preferably, concentrations of the primers and probe in a reaction system are as follows respectively: the forward primer has a concentration of 30 nM-1000 nM, the reverse primer has a concentration of 30 nM-500 nM, and the probe has a concentration of 150 nM-1200 nM.

In some embodiments, there is provided a probe primer composition for detecting respiratory syncytial virus (RSV) and influenza A virus (IAV), comprising a probe, forward primers, and reverse primers selected from the following group: a probe with a sequence shown in SEQ ID NO: 4;
a forward primer with a sequence shown in SEQ ID NO: 5;
a reverse primer with a sequence shown in SEQ ID NO: 6;
a forward primer with a sequence shown in SEQ ID NO: 7; and
a reverse primer with a sequence shown in SEQ ID NO: 8.

Preferably, the reaction conditions are as follows: performing reverse transcription at 35-65° C. for 5-60 minutes, and performing pre-denaturation at 90-96° C. for 5-15 minutes; performing denaturation at 90° C.-95° C. for 10-60 seconds, and performing annealing and extension at 50° C.-75° C. for 30-90 seconds, for 35-50 cycles; performing denaturation at 90° C.-95° C. for 10-600 seconds, and performing incubation at a constant temperature of 30° C.-55° C. for 10-600 seconds; and performing melting curve analysis at 35-95° C. with a heating rate of 0.05° C./s, and collecting fluorescence signals.

More preferably, the reaction conditions are as follows: performing reverse transcription at 55° C. for 15 minutes; performing pre-denaturation at 95° C. for 2 minutes; performing denaturation at 94° C. for 10 seconds, and performing annealing and extension at 56° C. for 30 seconds, for a total of 45 cycles; performing denaturation at 94° C. for 150 seconds, and performing incubation at a constant temperature of 40° C. for 300 seconds; and performing melting curve analysis at 40-95° C. with a heating rate of 0.05° C./S, and collecting fluorescence signals.

Preferably, concentrations of the primers and probe in a reaction system of the present invention are as follows respectively: the forward primer (F) has a concentration of 30 nM-1000 nM, the reverse primer (R) has a concentration of 30 nM-500 nM, and the probe (P) has a concentration of 150 nM-1200 nM.

In some embodiments, the first primer further comprises an amplification product capturing region B, and the probe primer composition further comprises a second primer set, wherein the second primer set comprises a third primer and the second primer that are a forward primer and a reverse primer or a reverse primer and a forward primer, respectively, the third primer comprises a third target sequence binding region and an amplification product capturing region B, and the second primer further comprises a second target sequence binding region.

Preferably, the third primer sequentially comprises the amplification product capturing region B and the third target sequence binding region from 5′ end to 3′ end; the first primer sequentially comprises the sequence region A and the amplification product capturing region B from 5′ end to 3′ end, and the second primer sequentially comprises the sequence region h and the second target sequence binding region from 5′ end to 3′ end; and the amplification product capturing region B does not bind with the target sequence or any fragment thereof in a paired manner.

The second primer further comprises an extension blocking region, which is a sequence that is not identical to or complementary with a base sequence of any part of the probe.

More preferably, the second primer sequentially comprises the extension blocking region, the sequence region h, and the second target sequence binding region from 5′ end to 3′ end.

Preferably, the target sequence binding region is a target specific sequence, and specifically binds with a target sequence to be amplified.

In some embodiments, an annealing temperature between the probe and the first primer is TmA, an annealing temperature between the probe and a complementary sequence of the second primer is TmH, an annealing temperature between the third primer and the target sequence is Tmb1, and an annealing temperature between the second primer and the target sequence is Tmb2. In some specific embodiments, TmA<Tmb1 and TmA<Tmb2, and/or TmH<Tmb1 and TmH<Tmb2.

Preferably, the third primer has a length of 20-80 bases.

Preferably, the third target sequence binding region of the third primer has a Tm value of 40° C.-80° C.

Preferably, the third target sequence binding region of the third primer has a GC content of 20%-80%.

Preferably, the amplification product capturing region B has a length of 5-35 bases.

Preferably, the amplification product capturing region B has a Tm value of 40° C.-80° C.

Preferably, the amplification product capturing region B has a GC content of 20%-80%.

Preferably, the first primer has a length of 20-80 bases, wherein the sequence region A has a length of 5-65 bases.

Preferably, the sequence region A of the first primer has a Tm value of 40° C.-80° C.

Preferably, the sequence region A of the first primer has a GC content of 30%-80%.

Preferably, there may be 0-20 bases spaced between the amplification product capturing region B and the target sequence binding region.

In some embodiments, when there is no target to be amplified, the first binding region A′ of the probe complementarily binds with the sequence region A of the first primer, and the other part of the probe is in a single-stranded state, the single-stranded flexible probe is formed as a curly shape due to molecular flexibility, at this time, a distance between the first detectable label and the second detectable label is relatively close, and the efficiency of fluorescence resonance energy transfer is relatively high.

In some embodiments, during PCR amplification, the third primer and the second primer respectively specifically bind with the target sequence and extend to produce a first double-stranded amplification product. One of first single-stranded amplification products of the first double-stranded amplification product comprises the extension blocking region, the sequence region h, the target sequence, and a complementary sequence of the amplification product capturing region B; then, the amplification product capturing region B of the first primer is complementary with the complementary sequence of the amplification product capturing region at 3′ end of the first single-stranded amplification product and extends to obtain a second double-stranded amplification product. One of second single-stranded amplification products of the second double-stranded amplification product comprises the sequence region A, the amplification product capturing region B, the target sequence, a complementary sequence of the sequence region h, and a complementary sequence of the extension blocking region.

In such embodiments, the second binding region H of the probe is complementarily paired with the complementary sequence of the sequence region h of the second single-stranded amplification product, and the first binding region A′ of the probe is complementarily paired with the sequence region A of the second single-stranded amplification product. At this time, the probe is changed from a single-stranded state to a double-stranded state, and a distance between the first detectable label and the second detectable label is increased, thereby generating a signal that may be detected by an instrument.

In some embodiments, both of the first detectable label and the second detectable label of the probe are located on the first binding region A′; or both of the first detectable label and the second detectable label of the probe are located on the second binding region H; or a spacer region between the first detectable label and the second detectable label is at least partially located on the first binding region A′; or a spacer region between the first detectable label and the second detectable label is at least partially located on the second binding region H. The second binding region H of the probe is complementarily paired with the complementary sequence of the sequence region h of the second single-stranded amplification product, and the first binding region A′ of the probe is complementarily paired with the sequence region A of the second single-stranded amplification product, the second single-stranded pre-amplification product forms an omega (C) hybridization form or an open-loop hybridization form with the probe, and the distance between the first detectable label and the second detectable label is increased, thereby generating a signal that may be detected by the instrument.

Preferably, the complementary sequence of the extension blocking region of the first single-stranded amplification product is not complementary with the probe and any fragment thereof.

In some embodiments, a target sequence amplification is identified by means of a fluorescence channel, or a target sequence amplification is identified by means of a melting temperature curve, or a target sequence amplification is identified by means of a fluorescence channel+a melting temperature curve.

Preferably, a target sequence amplification is identified by means of the melting temperature curve. Specifically, after PCR amplification, a melting curve analysis is performed, during a process of temperature change, the double-stranded product formed by the probe and the second single-stranded amplification product will melt at a certain temperature, at this time, the probe with the first detectable label and the second detectable label will be changed from a double strand to a single strand, the distance between the first detectable label and the second detectable label is decreased and the efficiency of fluorescence resonance energy transfer is increased, thereby generating a signal that may be detected by the instrument.

In some embodiments, the probe primer composition comprises n second primer sets, m probes and o first primers for n target sequences, wherein 1≤n≤20, 1≤m≤n, and 1≤o≤n; and preferably, wherein 1≤m≤o.

Single-stranded amplification products obtained by amplification with all of the first primer sets for the same probe are different from each other in an annealing temperature with the probe.

Preferably, single-stranded amplification products obtained by amplification with all of the first primer sets for the same probe are different from each other by a ΔTm of 6-10° C. in an annealing temperature with the probe Preferably, the target sequence binding regions included in all of the third primers are different from each other, and the target sequence binding regions included in all of the second primers are different from each other.

Preferably, the probe primer composition for n target sequences comprises n second primer sets, 1 or several probes, and 1 or several first primers, wherein 1≤n≤20.

In some embodiments, n probes and n first primers are designed for n target sequences, and then an identification of amplifications of n target sequences is carried out by using a fluorescence channel. In such embodiments, the n probes are flexible probes.

The flexible probe forms a curly shape due to molecular flexibility in single-stranded state, but after it is extended to be double-stranded state by hybridization or annealing, it is fixed to form a relatively rigid double-helix structure due to hydrogen-bond interaction. The flexible probe may be selected from a group consisting of: an oligonucleotide that forms a curly shape due to molecular flexibility in single-stranded state, an oligonucleotide that can form a hairpin structure in single-stranded state, an oligonucleotide that can form a stem-loop structure in single-stranded state, an oligonucleotide that can form a pseudoknot structure in single-stranded state, and an oligonucleotide that can form a triplex structure in single-stranded state.

In some embodiments, only one probe and one first primer are designed for n target sequences, and then amplifications of n target sequences are identified by using melting curves. In such embodiments, a sequence and/or length of the sequence region h of the second primer is adjusted to obtain the sequence region A and the complementary sequence of the sequence region h of the second single-stranded pre-amplification product which are partially or completely complementary with the first binding region A′ and the second binding region H of the probe, such that a melting temperature of the double-stranded product formed by the probe and the second single-stranded amplification product is adjusted.

Preferably, a length or sequence of the sequence region A and/or the sequence region h is adjusted, or positions at which the sequence region A and/or the complementary sequence of the sequence region h are complementary with the first binding region A′ and/or the second binding region H of the probe are adjusted, the melting temperature of the double-stranded product formed by the probe and the second single-stranded amplification product may be increased or decreased.

Such embodiments can achieve super-multiplex PCR in a single tube and effectively avoid the formation of primer dimers.

In some embodiments, there is provided a probe primer composition for detecting respiratory syncytial virus, comprising:
a probe with a sequence shown in SEQ ID NO: 9;
a third primer with a sequence shown in SEQ ID NO: 17;
a first primer with a sequence shown in SEQ ID NO: 18; and
a second primer with a sequence shown in SEQ ID NO: 16.

Preferably, the reaction conditions are as follows: performing reverse transcription at 35-65° C. for 5-60 minutes, and performing pre-denaturation at 90-96° C. for 5-15 minutes; performing denaturation at 90° C.-95° C. for 10-60 seconds, and performing annealing and extension at 50° C.-75° C. for 30-90 seconds, for 35-50 cycles; performing denaturation at 90° C.-95° C. for 10-600 seconds, and performing incubation at a constant temperature of 30° C.-55° C. for 10-600 seconds, performing melting curve analysis at 35-95° C. with a heating rate of 0.05° C./s, and collecting fluorescence signals.

More preferably, the reaction conditions are as follows: performing reverse transcription at 55° C. for 15 minutes, and performing pre-denaturation at 95° C. for 2 minutes; performing denaturation at 94° C. for 10 seconds, and performing annealing and extension at 56° C. for 30 seconds, for a total of 45 cycles; performing denaturation at 94° C. for 150 seconds, and performing incubation at a constant temperature of 40° C. for 300 seconds; and performing melting curve analysis at 40-95° C. with a heating rate of 0.05° C./S, and collecting fluorescence signals.

Preferably, concentrations of the primers and probe in a reaction system of the present invention are as follows respectively: the third primer (F) has a concentration of 30 nM-1000 nM, the second primer (R) has a concentration of 30 nM-500 nM, the first primer (F0) has a concentration of 30 nM-1000 nM, and the probe (P) has a concentration of 150 nM-1200 nM.

In some embodiments, the second primer further comprises an amplification product capturing region C, and the probe primer composition further comprises a second primer set, the second primer set comprises the first primer and a fourth primer that are a forward primer and a reverse primer or a reverse primer and a forward primer, respectively, wherein the fourth primer set comprises a fourth target sequence binding region and an amplification product capturing region C, and the first primer further comprises a first target sequence binding region.

In some embodiments, the first primer sequentially comprises the sequence region A and the first target sequence binding region from 5′ end to 3′ end; the second primer sequentially comprises the sequence region h and the amplification product capturing region C from 5′ end to 3′ end; and the fourth primer sequentially comprises the amplification product capturing region C and the fourth target sequence binding region from 5′ end to 3′ end,

The amplification product capturing region C does not bind with a target sequence or any fragment thereof in a paired manner.

Preferably, the second primer further comprises an extension blocking region, which is a sequence that is not identical to or complementary with a base sequence of any part of the probe.

More preferably, the second primer sequentially comprises the extension blocking region, the sequence region h, and the amplification product capturing region C from 5′ end to 3′ end.

In some embodiments, an annealing temperature between the probe and the first primer is TmA, an annealing temperature between the probe and a complementary sequence of the second primer is TmH, an annealing temperature between the first primer and the target sequence is Tmb1, and an annealing temperature between the fourth primer and the target sequence is Tmb2. In some specific embodiments, TmA<Tmb1 and TmA<Tmb2, and/or TmH<Tmb1 and TmH<Tmb2.

Preferably, the fourth primer has a length of 20-80 bases.

Preferably, the fourth target sequence binding region of the fourth primer has a Tm value of 40° C.-80° C.

Preferably, the fourth target sequence binding region of the fourth primer has a GC content of 20%-80%.

Preferably, the amplification product capturing region C is has a length of 5-35 bases.

Preferably, the amplification product capture region C has a Tm value of 40° C.-80° C.

Preferably, the amplification product capturing region C has a GC content of 20%-80%.

Preferably, the first primer has a length of 20-80 bases, with sequence region A having a length of 5-65 bases.

Preferably, the sequence region A of the first primer has a Tm value of 40° C.-80° C.

Preferably, the sequence region A of the first primer has a GC content of 30%-80%.

Preferably, there may be a 0-20 bases spaced between the amplification product capture region C and the target sequence binding region.

In some embodiments, when there is no target to be amplified, the first binding region A′ of the probe complementarily binds with the sequence region A of the first primer, and the other part of the probe is in a single-stranded state, the single-stranded flexible probe is formed as a curly shape due to molecular flexibility, at this time, a distance between the first detectable label and the second detectable label is relatively close, and the efficiency of fluorescence resonance energy transfer is relatively high.

In some embodiments, during PCR amplification, the first primer and the fourth primer of the second primer set are used to specifically bind with a target sequence and extend to produce a first double-stranded pre-amplification product, wherein one single-stranded amplification product of the first double-stranded pre-amplification product comprises a complementary sequence of the sequence region A, the target sequence, and the amplification product capture region C;
the first primer set is used and the single-stranded amplification product is taken as a template to extend to obtain a second double-stranded amplification product, wherein one single-stranded amplification product of the second double-stranded amplification product comprises the sequence region A, the target sequence, a complementary sequence of the amplification product capturing region C, and a complementary sequence of the sequence region h, and
a signal change that can be detected by an instrument is formed, by specifical binding of the A and h′ of the single-stranded amplification product with the probe, and whether the target sequence is present is determined based on the signal change.

Preferably, the one single-stranded amplification product of the first double-stranded pre-amplification product and/or the one single-stranded amplification product of the second double-stranded amplification product further comprises a complementary sequence of the extension blocking region, and the complementary sequence of the extension blocking region is not complementary with any part of the probe.

In such embodiments, the second binding region H of the probe is complementarily paired with the complementary sequence of the sequence region h of the second single-stranded amplification product, and the first binding region A′ of the probe is complementarily paired with the sequence region A of the second single-stranded amplification product. At this time, the probe is changed from a single-stranded state to a double-stranded state, and a distance between the first detectable label and the second detectable label is increased, thereby generating a signal that may be detected by an instrument.

In some embodiments, both of the first detectable label and the second detectable label of the probe are located on the first binding region A′; or both of the first detectable label and the second detectable label of the probe are located on the second binding region H; or a spacer region between the first detectable label and the second detectable label is at least partially located on the first binding region A′; or a spacer region between the first detectable label and the second detectable label is at least partially located on the second binding region H. The second binding region H of the probe is complementarily paired with the complementary sequence of the sequence region h of the second single-stranded amplification product, and the first binding region A′ of the probe is complementarily paired with the sequence region A of the second single-stranded amplification product, the second single-stranded amplification product forms an omega (O) hybridization form or an open-loop hybridization form with the probe, and the distance between the first detectable label and the second detectable label is increased, thereby generating a signal that may be detected by the instrument.

Preferably, the complementary sequence of the extension blocking region of the second single-stranded amplification product is not complementary with the probe and any fragment thereof.

In some embodiments, a target sequence amplification is identified by means of a fluorescence channel, or a target sequence amplification is identified by means of a melting temperature curve, or a target sequence amplification is identified by means of a fluorescence channel+a melting temperature curve.

Preferably, a target sequence amplification is identified by means of the melting temperature curve. Specifically, after PCR amplification, a melting curve analysis is performed, during a process of temperature change, the double-stranded product formed by the probe and the second single-stranded amplification product will melt at a certain temperature, at this time, the probe with the first detectable label and the second detectable label will be changed from a double strand to a single strand, the distance between the first detectable label and the second detectable label is decreased and the efficiency of fluorescence resonance energy transfer is increased, thereby generating a signal that may be detected by the instrument.

In some embodiments, the probe primer composition for n target sequences comprises n second primer sets, m probes and o second primers, wherein 1≤n≤20, 1≤m≤n, and 1≤o≤n; and preferably, wherein 1≤m≤o.

Single-stranded amplification products obtained by amplification with all of the first primer sets for the same probe are different from each other in an annealing temperature with the probe.

Preferably, single-stranded amplification products obtained by amplification with all of the first primer sets for the same probe are different from each other by a ΔTm of 6-10° C. in an annealing temperature with the probe

Preferably, the target sequence binding regions included in all of the first primers are different from each other, and the target sequence binding regions included in all of the fourth primers are different from each other.

Preferably, the probe primer composition comprises n second primer sets, 1 or several probes, and 1 or several second primers for n target sequences, wherein 1≤n≤20.

In some embodiments, n probes and n second primers are designed for n target sequences, and then an identification of amplifications of n target sequences is carried out by using a fluorescence channel. In such embodiments, the n probes are flexible probes.

The flexible probe forms a curly shape due to molecular flexibility in single-stranded state, but after it is extended to be double-stranded state by hybridization or annealing, it is fixed to form a relatively rigid double-helix structure due to hydrogen-bond interaction. The flexible probe may be selected from a group consisting of: an oligonucleotide that forms a curly shape due to molecular flexibility in single-stranded state, an oligonucleotide that can form a hairpin structure in single-stranded state, an oligonucleotide that can form a stem-loop structure in single-stranded state, an oligonucleotide that can form a pseudoknot structure in single-stranded state, and an oligonucleotide that can form a triplex structure in single-stranded state.

In some embodiments, only one probe and one second primer are designed for n target sequences, and then amplifications of n target sequences are identified by using melting curves. In such embodiments, a sequence and/or length of the sequence region A of the first primer is adjusted to obtain the sequence region A and the complementary sequence of the sequence region h of the second single-stranded pre-amplification product which are partially or completely complementary with the first binding region A′ and the second binding region H of the probe, such that a melting temperature of the double-stranded product formed by the probe and the first single-stranded amplification product is adjusted.

Preferably, a length or sequence of the sequence region A and/or the sequence region h is adjusted, or positions at which the sequence region A and/or the complementary sequence of the sequence region h are complementary with the first binding region A′ and/or the second binding region H of the probe are adjusted, the melting temperature of the double-stranded product formed by the probe and the second single-stranded amplification product may be increased or decreased.

Such embodiments can achieve super-multiplex PCR in a single tube and effectively avoid the formation of primer dimers.

In some embodiments, the first primer further comprises an amplification product capturing region B, the second primer further comprises an amplification product capturing region C, and the probe primer composition further comprises a second primer set, the second primer set comprises a third primer and a fourth primer that are a forward primer and a reverse primer or a reverse primer and a forward primer, respectively, the third primer set comprises a third target sequence binding region and an amplification product capturing region B, and the fourth primer set comprises a fourth target sequence binding region and an amplification product capturing region C.

In some embodiments, the first primer sequentially comprises the sequence region A and the amplification product capturing region B from 5′ end to 3′ end; the second primer sequentially comprises the sequence region h and the amplification product capturing region C from 5′ end to 3′ end; the third primer sequentially comprises the amplification product capturing region B and the third target sequence binding region from 5′ end to 3′ end; and the fourth primer sequentially comprises the amplification product capturing region C and the fourth target sequence binding region from 5′ end to 3′ end.

Neither the amplification product capturing region B nor the amplification product capturing region C is paired with the target sequence or any fragment thereof.

Preferably, the second primer further comprises an extension blocking region, which is a base sequence that is not identical to or complementary with the probe or any fragment thereof.

More preferably, the second primer sequentially comprises the extension blocking region, the sequence region h, and the amplification product capturing region C from 5′ end to 3′ end.

Preferably, the third target sequence binding region of the third primer is a target specific sequence, and specifically binds with the target sequence to be amplified.

An annealing temperature between the probe and the first primer is TmA, an annealing temperature between the probe and a complementary sequence of the second primer is TmH, an annealing temperature between the third primer and the target sequence is Tmb1, and an annealing temperature between the fourth primer and the target sequence is Tmb2. In some specific embodiments, TmA<Tmb1 and TmA<Tmb2, and/or TmH<Tmb1 and TmH<Tmb2 Preferably, the third primer comprises the amplification product capturing region B and the third target sequence binding region.

Preferably, the third primer has a length of 20-80 bases.

Preferably, the third target sequence binding region of the third primer has a Tm value of 40° C.-80° C.

Preferably, the third target sequence binding region of the third primer has a GC content of 20%-80%.

Preferably, the amplification product capturing region B has a length of 5-35 bases.

Preferably, the amplification product capturing region B has a Tm value of 40° C.-80° C.

Preferably, the amplification product capturing region B has a GC content of 20%-80%.

Preferably, the first primer has a length of 20-80 bases, with sequence region A having a length of 5-35 bases.

Preferably, the sequence region A of the first primer has a Tm value of 40° C.-80° C.

Preferably, the sequence region A of the first primer has a GC content of 30%-80%.

Preferably, there may be a 0-20 bases spaced between the amplification product capturing region B and the target sequence binding region.

Preferably, the second primer further comprises the extension blocking region, which is a freely designed sequence that is not identical to a base sequence of any part of the probe.

Preferably, the fourth target sequence binding region of the fourth primer is a target specific sequence, and specifically binds with the target sequence to be amplified.

Preferably, the fourth primer comprises an amplification product capturing region C and a fourth target sequence binding region.

Preferably, the fourth primer has a length of 20-80 bases.

Preferably, the fourth target sequence binding region of the fourth primer has a Tm value of 40° C.-80° C.

Preferably, the fourth target sequence binding region of the fourth primer has a GC content of 20%-80%.

Preferably, the amplification product capturing region C has a length of 5-35 bases.

Preferably, the amplification product capture region C has a Tm value of 40° C.-80° C.

Preferably, the amplification product capturing region C has a GC content of 20%-80%.

Preferably, the second primer comprises the extension blocking region, the sequence region h, and the amplification product capturing region C.

Preferably, the second primer has a length of 20-80 bases, with sequence region h having a length of 5-65 bases.

Preferably, the sequence region h of the second primer has a Tm value of 30-80° C.

Preferably, the sequence region h of the second primer has a GC content of 20-80%.

Preferably, the extension blocking region of the second primer has a length of 1-20 bp.

Preferably, there are 0-20 bases spaced between the sequence region h and the amplification product capture region C.

In some embodiments, when there is no target to be amplified, the first binding region A′ of the probe complementarily binds with the sequence region A of the first primer, and the other part of the probe is in a single-stranded state, the single-stranded flexible probe is formed as a curly shape due to molecular flexibility, at this time, a distance between the first detectable label and the second detectable label is relatively close, and the efficiency of fluorescence resonance energy transfer is relatively high.

In some embodiments, during a first PCR amplification, the third primer and the fourth primer specifically bind with a target sequence respectively and extend to produce a first double-stranded amplification product, wherein one of single-stranded amplification products of the first double-stranded amplification product comprises the amplification product capturing region B, the target sequence, and a complementary sequence of the amplification product capturing region C; then, the first double-stranded amplification product is taken as a template, and the first primer and the second primer are used to perform a second PCR to obtain a second double-stranded amplification product, wherein one of second single-stranded amplification products of the second double-stranded amplification product comprises the sequence region A, the amplification product capturing region B, the target sequence, a complementary sequence of the amplification product capturing region C, a complementary sequence of the sequence region h, and a complementary sequence of the extension blocking region.

In such embodiments, the second binding region H of the probe is complementarily paired with the complementary sequence of the sequence region h of the second single-stranded amplification product, and the first binding region A′ of the probe is complementarily paired with the sequence region A of the second single-stranded amplification product at 5′ end thereof. At this time, the probe is changed from a single-stranded state to a double-stranded state, and a distance between the first detectable label and the second detectable label is increased, thereby generating a signal that may be detected by an instrument.

In some embodiments, both of the first detectable label and the second detectable label of the probe are located on the first binding region A′; or both of the first detectable label and the second detectable label of the probe are located on the second binding region H; or a spacer region between the first detectable label and the second detectable label is at least partially located on the first binding region A′; or a spacer region between the first detectable label and the second detectable label is at least partially located on the second binding region H. The second binding region H of the probe is complementarily paired with the complementary sequence of the sequence region h of the second single-stranded amplification product, and the first binding region A′ of the probe is complementarily paired with the sequence region A of the second single-stranded amplification product, the second single-stranded amplification product forms an omega (Ω) hybridization form or an open-loop hybridization form with the probe, and the distance between the first detectable label and the second detectable label is increased, thereby generating a signal that may be detected by the instrument.

Preferably, the complementary sequence of the extension blocking region of the second single-stranded pre-amplification product at 3′ end thereof is not complementary with the probe and any fragment thereof.

In some embodiments, a target sequence amplification is identified by means of a fluorescence channel, or a target sequence amplification is identified by means of a melting temperature curve, or a target sequence amplification is identified by means of a fluorescence channel+a melting temperature curve.

Preferably, a target sequence amplification is identified by means of the melting temperature curve. Specifically, after the second PCR amplification, a melting curve analysis is performed, during a process of temperature change, the double-stranded product formed by the probe and the second single-stranded amplification product will melt at a certain temperature, at this time, the probe with the first detectable label and the second detectable label will be changed from a double strand to a single strand, such that the distance between the first detectable label and the second detectable label is decreased and the efficiency of fluorescence resonance energy transfer is increased, thereby generating a signal that may be detected by the instrument.

In some embodiments, the probe primer composition comprises n second primer sets for n target sequences, m probes and p first primer sets, wherein 1≤n≤20, 1≤m≤n, and 1≤p≤n.

Single-stranded amplification products obtained by amplification with all of the first primer sets for the same probe are different from each other in an annealing temperature with the probe.

Preferably, single-stranded amplification products obtained by amplification with all of the first primer sets for the same probe are different from each other by a ΔTm of 6-10° C. in an annealing temperature with the probe Preferably, the target sequence binding regions included in all of the third primers are different from each other, and the target sequence binding regions included in all of the fourth primers are different from each other.

Preferably, the probe primer composition for n target sequences comprises n second primer sets, 1 or several probes, and 1 or several first primer sets, wherein 1≤n≤20.

In some embodiments, n probes, n first primers and n second primers are designed for n target sequences, and then an identification of amplifications of n target sequences is carried out by using a fluorescence channel. In such embodiments, the n probes are flexible probes.

The flexible probe forms a curly shape due to molecular flexibility in single-stranded state, but after it is extended to be double-stranded state by hybridization or annealing, it is fixed to form a relatively rigid double-helix structure due to hydrogen-bond interaction. The flexible probe may be selected from a group consisting of: an oligonucleotide that forms a curly shape due to molecular flexibility in single-stranded state, an oligonucleotide that can form a hairpin structure in single-stranded state, an oligonucleotide that can form a stem-loop structure in single-stranded state, an oligonucleotide that can form a pseudoknot structure in single-stranded state, and an oligonucleotide that can form a triplex structure in single-stranded state.

In some embodiments, only one probe, one first primer and one second primer are designed for n target sequences, and then amplifications of n target sequences are identified by using melting curves. In such embodiments, a sequence and/or length of the sequence region A of the first primer and/or the sequence region h of the second primer are adjusted to obtain the sequence region A and the complementary sequence of the sequence region h of the second single-stranded pre-amplification product which are partially or completely complementary with the first binding region A′ and the second binding region H of the probe, such that a melting temperature of the double-stranded product formed by the probe and the second single-stranded amplification product is adjusted.

Preferably, a length or sequence of the sequence region A and/or the sequence region h is adjusted, or positions at which the sequence region A and/or the complementary sequence of the sequence region h are complementary with the first binding region A′ and/or the second binding region H of the probe are adjusted, the melting temperature of the double-stranded product formed by the probe and the second single-stranded amplification product may be increased or decreased.

Such embodiments can achieve super-multiplex PCR in a single tube and effectively avoid the formation of primer dimers.

In some embodiments, there is provided a probe primer composition for detecting respiratory syncytial virus, comprising:
a probe with a sequence shown in SEQ ID NO: 9;
a third primer with a sequence shown in SEQ ID NO: 17;
a first primer with a sequence shown in SEQ ID NO: 18;
a fourth primer with a sequence shown in SEQ ID NO: 19; and
a second primer with a sequence shown in SEQ ID NO: 20.

Preferably, the reaction conditions are as follows: performing reverse transcription at 35-65° C. for 5-60 minutes, and performing pre-denaturation at 90-96° C. for 5-15 minutes; performing denaturation at 90° C.-95° C. for 10-60 seconds, and performing annealing and extension at 50° C.-75° C. for 30-90 seconds, for 35-50 cycles; performing denaturation at 90° C.-95° C. for 10-600 seconds, and performing incubation at a constant temperature of 30° C.-55° C. for 10-600 seconds, performing melting curve analysis at 35-95° C. with a heating rate of 0.05° C./s, and collecting fluorescence signals.

More preferably, the reaction conditions are as follows: performing reverse transcription at 55° C. for 15 minutes; performing pre-denaturation at 95° C. for 2 minutes; performing denaturation at 94° C. for 10 seconds, and performing annealing and extension at 56° C. for 30 seconds, for a total of 45 cycles; performing denaturation at 94° C. for 150 seconds, and performing incubation at a constant temperature of 40° C. for 300 seconds; and performing melting curve analysis at 40-95° C. with a heating rate of 0.05° C./S, and collecting fluorescence signals.

Preferably, concentrations of the primers and probe in a reaction system of the present invention are as follows respectively: the third primer (F) has a concentration of 30 nM-1000 nM, the fourth primer (R) has a concentration of 30 nM-500 nM, the first primer (F0) has a concentration of 30 nM-1000 nM, the second primer (F) has a concentration of 30 nM-1000 nM, and the probe (P) has a concentration of 150 nM-1200 nM.

In a second aspect, the present invention provides a reaction system for PCR detection, comprising the probe primer composition of the first aspect.

In a third aspect, the present invention provides a kit for PCR detection, comprising the probe primer composition of the first aspect or the reaction system of the second aspect.

In a fourth aspect, the present invention provides a method for PCR detection, comprising steps of performing PCR by using the probe primer composition of the first aspect, the reaction system of the second aspect, or the kit of the third aspect.

In some embodiments, the first primer set is used to specifically bind with a target sequence and extend to produce a double-stranded pre-amplification product, wherein one single-stranded amplification product of the double-stranded pre-amplification product comprises the sequence region A, the target sequence, and a complementary sequence h′ of the sequence region h; and
a signal change that can be detected by an instrument is formed, by specifical binding of the A and h′ of the single-stranded amplification product with the probe, and whether the target sequence is present is determined based on the signal change.

Preferably, the single-stranded amplification product further comprises a complementary sequence of the extension blocking region at 3′ end thereof, which is not complementary with any part of the probe.

In some embodiments, the third primer and the second primer of the second primer set are used to specifically bind with a target sequence and extend to produce a first double-stranded pre-amplification product, wherein one single-stranded amplification product of the first double-stranded pre-amplification product comprises a complementary sequence B′ of the amplification product capturing region B, the target sequence, and the sequence region h;
the first primer set is used and the first double-stranded pre-amplification product is taken as a template, to extend to obtain a second double-stranded amplification product, wherein one single-stranded amplification product of the second double-stranded amplification product comprises the sequence region A, the amplification product capturing region B, the target sequence, and a complementary sequence h′ of the sequence region h; and
a signal change that can be detected by an instrument is formed, by specifical binding of the A and h′ of the single-stranded amplification product with the probe, and whether the target sequence is present is determined based on the signal change.

Preferably, the one single-stranded amplification product of the first double-stranded pre-amplification product, and/or the one single-stranded amplification product of the second double-stranded amplification product further comprises a complementary sequence of the extension blocking region, and the complementary sequence of the extension blocking region is not complementary with any part of the probe.

In some embodiments, the first primer and the fourth primer of the second primer set are used to specifically bind with a target sequence and extend to produce a first double-stranded pre-amplification product, wherein one single-stranded amplification product of the first double-stranded pre-amplification product comprises a complementary sequence of the sequence region A, the target sequence, and the amplification product capturing region C;
the first primer set is used and the first double-stranded pre-amplification product is taken as a template to extend to obtain a second double-stranded amplification product, wherein one single-stranded amplification product of the second double-stranded amplification product comprises the sequence region A, the target sequence, a complementary sequence of the amplification product capturing region C, and a complementary sequence h′ of the sequence region h, and
a signal change that can be detected by an instrument is formed, by specifical binding of the A and h′ of the single-stranded amplification product with the probe, and whether the target sequence is present is determined based on the signal change.

Preferably, the one single-stranded amplification product of the second double-stranded amplification product further comprises a complementary sequence of the extension blocking region, and the complementary sequence of the extension blocking region is not complementary with any part of the probe.

In some embodiments, the third primer and the fourth primer of the second primer set are used to specifically bind with a target sequence and extend to produce a first double-stranded pre-amplification product, wherein one single-stranded amplification product of the first double-stranded pre-amplification product comprises a complementary sequence of the sequence B, the target sequence, and the amplification product capturing region C;
the first primer set is used and the first double-stranded pre-amplification product is taken as a template to extend to obtain a second double-stranded amplification product, wherein one single-stranded amplification product of the second double-stranded amplification product comprises the sequence region A, the amplification product capturing region B, the target sequence, a complementary sequence of the amplification product capturing region C, and a complementary sequence h′ of the sequence region h; and
a signal change that can be detected by an instrument is formed, by specifical binding of the A and h′ of the single-stranded amplification product with the probe, and whether the target sequence is present is determined based on the signal change.

Preferably, the one single-stranded amplification product of the second double-stranded amplification product further comprises a complementary sequence of the extension blocking region, and the complementary sequence of the extension blocking region is not complementary with any part of the probe.

In a fifth aspect, the present invention provides a use of the probe primer composition of the first aspect, the reaction system of the second aspect, or the kit of the third aspect in PCR detection.

In particular, the present invention provides a use of the probe primer composition of the first aspect, the reaction system of the second aspect, or the kit of the third aspect in single-tube super-multiplex PCR detection.

In a sixth aspect, the present invention provides a use of the probe primer composition of the first aspect, the reaction system of the second aspect, the kit of the third aspect, or the method of the fourth aspect in pathogenic microorganism detection, non-invasive birth defect screening, single nucleotide polymorphism analysis, methylation analysis, gene mutation analysis, or gene typing.

### BRIEF DESCRIPTION OF THE DRAWINGS

FIG. 1 shows a primer probe design principle and method in a preferred embodiment 1 of the present invention.

FIG. 2 shows a primer probe design principle and method in a preferred embodiment 2 of the present invention.

FIG. 3 shows a primer probe design principle and method in a preferred embodiment 3 of the present invention.

FIG. 4 shows a melting curve representing RSV (62.25° C.) positive in a CY5 channel according to a singleplex PCR detection reaction of Example 1.

FIG. 5 is a view of an effect for detecting multiplex upper respiratory tract pathogens on a SLAN real-time fluorescence quantitative PCR instrument.

FIG. 6 is a view of an effect for detecting multiplex upper respiratory tract pathogens on a SLAN real-time fluorescence quantitative PCR instrument.

FIG. 7 shows melting curves representing RSV (62.38° C.) positive and a primer dimer (without melting peak) when a probe P5 is used in a CY5 channel according to a singleplex PCR detection reaction of Comparative Example.

FIG. 8 shows melting curves representing RSV positive (68.48° C., a melting peak with a high Tm value) and a primer dimer (68.52° C., a melting peak with a low Tm value) when a probe P4 is used in an FAM channel according to a singleplex PCR detection reaction of Comparative Example.

FIG. 9 shows a melting curve representing RSV (56.62° C.) positive in an FAM channel according to a singleplex PCR detection reaction of Example 3.

FIG. 10 shows a melting curve representing RSV (56.36° C.) positive in an FAM channel according to a singleplex PCR detection reaction of Example 4.

FIG. 11 illustrates a reaction principle of Example 5.

FIG. 12 shows that a melting peak with a Tm value of 56.76° C. is formed in a PCR reaction well with addition of non small cell lung cancer EGFR exon 20 H773_V774insH mutation positive sample in Example 5.

FIG. 13 illustrates a reaction principle of Example 6.

FIG. 14 shows that a melting peak with a Tm value of 53.67° C. is formed by reverse complementation of a probe anchoring region (A) of a forward primer (F9) and a primer anchoring region (A′) of a probe (P7) in a PCR reaction well in a CY5 channel in Example 6.

FIG. 15 shows that a melting peak with a Tm value of 52.43° C. and a positive melting peak with a Tm value of 62.41° C. are formed by reverse complementation of a probe anchoring region (A) of a forward primer (F9) and a primer anchoring region (A′) of a probe (P7) in a PCR reaction well with addition of non small cell lung cancer EGFR exon 20 H773_V774insH mutation positive sample in a CY5 channel.

FIG. 16 shows multiplex melting peaks of non-small cell lung cancer EGFR exon 20 T790M mutation, exon 21 L858R mutation, and exon 21 L861Q mutation in Example 7.

### DETAILED DESCRIPTION OF THE EMBODIMENTS

Preferred embodiments of the present invention are described in the following, but the protection scope of the present invention is not limited to the following preferred embodiments. It should be noted that, several variations and improvements made by those skilled in the art based on the concept of the present invention are within the protection scope of the present invention.

Herein, the terms “target sequence”, “target nucleic acid”, “target nucleic acid sequence”, or “target” may be used interchangeably and mean a portion of the nucleic acid sequence to be amplified, detected, or amplified and detected, which may be annealed with a primer under annealing or amplification conditions.

Herein, the term “upstream primer”, also known as forward primer, is an oligonucleotide that extends continuously along a negative strand; and the term “downstream primer” used in the present invention, also known as reverse primer, is an oligonucleotide that is continuously extended along a positive strand, wherein, the positive strand, i.e. a sense strand, also known as a positive-sense strand or a coding strand, is generally located at an upper end of a double-stranded DNA, with a direction of 5′-3′ from left to right, a base sequence thereof is substantially the same as a gene mRNA, and a primer binding with this strand is a reverse primer; and the negative strand, i.e. a nonsense strand, also known as a non-coding chain, is complementary with the positive strand, and a primer binding with this strand is a positive primer. It should be understood that, when the designations of the sense strand and the antisense strand are interchanged, the nominations of corresponding forward and reverse primer may also be interchanged accordingly.

Herein, the term “probe” refers to a labeled oligonucleotide for detecting whether a target is present.

Herein, the term “base-complementary pairing” refers to a phenomenon where A and T, A and U, and G and C, etc., in a corresponding relationship, are connected to each other via a hydrogen bond. Correspondingly, “mismatched bases” refer to all other pairing situations except for those specified in “base-complementary pairing”, such as a mismatching of A and C, A and G, T and G, or T and C.

### Embodiment 1

In this embodiment, the probe primer composition comprises a probe, a forward primer, and a reverse primer. The forward primer (F) sequentially comprises a probe anchoring region (A) and a target sequence binding region from 5′ end to 3′ end, wherein the target sequence binding region is a target specific sequence; and the probe anchoring region (A) is a freely designed sequence that does not bind with any target sequence in a paired manner and has a complementary with that of a primer anchoring region (A′) of the probe (P); and there are 0-20 bases spaced between the probe anchoring region (A) and the target sequence binding region.

The probe (P) is a freely designed sequence that does not bind with any target sequence in a paired manner, and comprises the primer anchoring region (A′) and a probe signal detection region (H); and the primer anchoring region (A′) is complementary with the probe anchoring region (A) of the forward primer (F).

In some specific embodiments, a first detectable label is labeled at a 1^{st}-95^{th} base position away from the primer anchoring region (A′), and a second detectable label is labeled at the base position spaced from the first detectable label by 5-25 bases.

In a specific embodiment, both of the first detectable label and the second detectable label are located in the primer anchoring region (A′) or in the probe signal detection region (H), wherein the first detectable label may be a fluorescence reporter, and the second detectable label may be a quencher or other labeling groups that can produce a signal change with the first detectable label via fluorescence resonance energy transfer (FRET).

The reverse primer (R) sequentially comprises an extension blocking region (M), a primer signal detection region (h), and a target sequence binding region from 5′ end to 3′ end; wherein the extension blocking region (M) is a freely designed sequence that is not identical to or complementary with a base segment of any portion of the probe (P); the target sequence binding region is a target specific sequence; the primer signal detection region (h) is a freely designed sequence that does not bind with any target sequence in a paired manner, and has a sequence partially or completely identical to that of the second binding region (H) of probe (P); an there may be 0-20 bases spaced between the primer signal detection region (h) and the target sequence binding region.

During PCR amplification, the forward primer (F) and reverse primer (R) specifically bind with a target sequence respectively and extend to produce a double-stranded amplification product, wherein one single-stranded amplification product (S1) sequentially comprises the probe anchoring region (A), the target sequence, a complementary sequence (h′) of the primer signal detection region, and a complementary sequence (M′) of the extension blocking region from 5′ end to 3′ end.

After the double-stranded amplification product is produced, the probe signal detection region (H) of the probe (P) is complementarily paired with the complementary sequence (h′) of the primer signal detection region of the one of single-stranded amplification products (S1), the primer anchoring region (A′) of the probe (P) is complementarily paired with the probe anchoring region (A) at 5′ end of the single-stranded amplification product (S1), and the complementary sequence (M′) of the extension blocking region at 3′ end of the single-stranded amplification product (S1) is not complementary with any part of the probe (P); and at this time, the probe (P) is changed from a single-stranded state to a double-stranded state, and a distance between the first detectable label and the second detectable label is changed, thereby generating a signal that may be detected by an instrument.

After the PCR amplification is completed, a melting curve analysis is performed, wherein, during a process of temperature change, the double-stranded product formed by the probe (P) and the single-stranded amplification product (S1) will melt at a certain temperature, at this time, the probe (P) with the first detectable label and the second detectable label will be changed from the double-stranded state to the single-stranded state, so as to cause a change in the distance between the detectable labels, thereby generating a signal that may be detected by the instrument.

Due to the fact that a melting temperature of the double-stranded product formed by the probe (P) and the single-stranded amplification product (S1) is detected during melting curve analysis, it is possible to adjust the melting temperature of the double-stranded product formed by the probe (P) and the single-stranded amplification product (S1) by adjusting a sequence and length of the primer signal detection region (h) of the reverse primer (R) or adjusting a sequence and length of the probe anchoring region A of the forward primer (F) to obtain the single-stranded amplification product (S1) that is partially or completely complementary with the primer anchoring region (A′) of the probe (P) and is partially or completely complementary with the probe signal detection region (H) of the probe (P). For example, it is possible to increase or decrease the melting temperature of the double-stranded product formed by the probe (P) and the single-stranded amplification product (S1) by adjusting a length or sequence of the primer signal detection region (h), or adjusting a position at which the complementary sequence (h′) of the primer signal detection is complementary with the probe signal detection region (H) of the probe (P).

When a multiplex PCR detection is performed, there may be multigroups of forward primers (F) and reverse primers (R) targeting different target sequences, and different target sequences may be identified by means of fluorescence channels or melting temperatures; and when different target sequences are identified by means of fluorescence channels, different probes (P) may be used for different target sequences, and when different target sequences are identified by means of melting temperatures, a same probe (P) may be shared by different target sequences.

In some specific embodiments, the forward primer (F) has a length of 20-80 bases, wherein the target sequence binding region has a Tm value of 40° C.-80° C. and a GC content of 20%-80%; and the probe anchoring region (A) has a length of 5-65 bases, a Tm value of 40° C.-80° C., and a GC content of 30%-80%.

In some specific embodiments, the reverse primer (R) has a length of 20-80 bases, wherein the extension blocking region (M) has a length of 1-20 bp; the target sequence binding region has a Tm value of 40° C.-80° C., and a GC content of 20%-80%; and the primer signal detection region (h) has a length of 5-65 bases, a Tm value of 30° C.-80° C., and a GC content of 20%-80%.

In some specific embodiments, the probe (P) has a length of 20-100 bases, wherein the primer anchoring region (A′) has a length of 5-65 bases, a Tm value of 40° C.-80° C., and a GC content of 30%-80%; more preferably, the primer anchoring region (A′) is designed at 3′ end of the probe (P); and the probe signal detection region (H) has a length of 5-65 bases, a Tm value of 30° C.-80° C., and a GC content of 20%-80%.

An annealing temperature of the probe and forward primer is TmA, an annealing temperature of the probe and the complementary sequence of the reverse primer is TmH, an annealing temperature of the forward primer and the target sequence is Tmb1, and an annealing temperature of the reverse primer and the target sequence is Tmb2. In some specific embodiments, the TmA<Tmb1 and TmA<Tmb2, and/or TmH<Tmb1 and TmH<Tmb2.

When there is no target to be detected, the primer anchoring region (A′) of the probe (P) is complementary with the probe anchoring region (A) of the forward primer (F), and the other part of the probe (P) is in a single-stranded state, and the single-stranded flexible probe has a curly shape due to molecular flexibility; and at this time, a distance between the first detectable label and the second detectable label is relatively close, and the efficiency of fluorescence resonance energy transfer is relatively high. When a target to be detected is present, the forward primer (F) and the reverse primer (R) specifically bind with a target sequence respectively and extend to produce a double-stranded amplification product, wherein one single-stranded amplification product (S1) of the amplification product sequentially comprises the probe anchoring region (A), the target sequence, a complementary sequence (h′) of the primer signal detection region, and a complementary sequence (M′) of the extension blocking region from 5′ end to 3′ end; the probe signal detection region (H) on the probe (P) is complementarily paired with the complementary sequence (h′) of the primer signal detection region of the single-stranded amplification product (S1), the complementary sequence (M′) of the extension blocking region at 3′ end of the single-stranded amplification product (S1) is not complementary with any part of the probe (P), and the primer anchoring region (A′) of the probe (P) is complementarily paired with the probe anchoring region (A) at 5′ end of the single-stranded amplification product (S1); and at this time, the distance between the first detectable label and the second detectable label is increased due to a change from the single strand to the double strand, and the efficiency of fluorescence resonance energy transfer is decreased, such that a fluorescence signal change is produced and may be detected by an instrument. After the completion of PCR, when the melting curve analysis is performed, the double-stranded product formed by the probe (P) and the single-stranded amplification product (S1) will melt at a certain temperature, such that the probe (P) with the first detectable label and the second detectable label becomes a single-stranded state, at this time, the molecular flexibility is restored, the distance between the first detectable label and the second detectable label is decreased, and the efficiency of fluorescence resonance energy transfer is increased, such that a fluorescence signal change is produced again, which may be used for melting curve analysis.

### Embodiment 2

In this embodiment, the probe primer composition comprises a probe, a first forward primer, a second forward primer, and a reverse primer. The first forward primer (F) sequentially comprises an amplification product capturing region (B) and a target sequence binding region from 5′ end to 3′ end, wherein the target sequence binding region is a target specific sequence; the amplification product capturing region (B) is a freely designed sequence that does not bind with any target sequence in a paired manner; and there are 0-20 bases spaced between the amplification product capturing region (B) and the target sequence binding region. The second forward primer (F0) comprises a probe anchoring region (A) and an amplification product capturing region (B) from 5′ end to 3′ end.

The probe (P) is a freely designed sequence that does not bind with any target sequence in a paired manner, and comprises the primer anchoring region (A′) and a probe signal detection region (H); and the primer anchoring region (A′) is complementary with the probe anchoring region (A) of the second forward primer (F0).

In some specific embodiments, a first detectable label is labeled at a 1^{st}-95^{th} base position away from the primer anchoring region (A′), and a second detectable label is labeled at the base position spaced from the first detectable label by 5-25 bases. Both of the first detectable label and the second detectable label are located on a same side of the primer anchoring region (A′), wherein the first detectable label may be a fluorescence reporter, and the second detectable label may be a quencher or other labeling groups that can produce a signal change with the first detectable label via fluorescence resonance energy transfer (FRET).

In some specific embodiments, the reverse primer (R) sequentially comprises an extension blocking region (M), a primer signal detection region (h), and a target sequence binding region from 5′ end to 3′ end; wherein the extension blocking region (M) is a freely designed sequence that is not identical to or complementary with a base segment of any portion of the probe (P); the target sequence binding region is a target specific sequence; the primer signal detection region (h) is a freely designed sequence that does not bind with any target sequence in a paired manner, and has a sequence partially or completely identical to that of the second binding region (H) of probe (P); and there are 0-20 bases spaced between the primer signal detection region (h) and the target sequence binding region.

During PCR amplification, the first forward primer (F) and reverse primer (R) specifically bind with a target sequence respectively and extend to produce a first double-stranded amplification product, wherein one single-stranded amplification product (S1) sequentially comprises the extension blocking region (M), the primer signal detection region (h), the target sequence, and a complementary sequence (B′) of the amplification product capturing region from 5′ end to 3′ end; and the amplification product capturing region (B) of the second forward primer (F0) may be complementary with the complementary sequence (B′) of the amplification product capturing region at 3′ end of the first single-stranded amplification product (S1) and extend to obtain a second double-stranded amplification product, wherein one second single-stranded amplification product (D1) sequentially comprises the probe anchoring region (A), the amplification product capturing region (B), the target sequence, a complementary sequence (h′) of the primer signal detection region (h), and a complementary sequence (M′) of the extension blocking region from 5′ end to 3′ end.

After the second double-stranded amplification product is produced, the probe signal detection region (H) of the probe (P) is complementarily paired with the complementary sequence (h′) of the primer signal detection region of the one second single-stranded amplification products (D1), the primer anchoring region (A′) of the probe is complementarily paired with the probe anchoring region (A) at 5′ end of the second single-stranded amplification product (D1), and the complementary sequence (M′) of the extension blocking region at 3′ end of the single-stranded amplification product (S1) is not complementary with any part of the probe (P); and at this time, the probe (P) is changed from a single-stranded state to a double-stranded state, and a distance between the first detectable label and the second detectable label is changed, thereby generating a signal that may be detected by an instrument.

After the PCR amplification is completed, a melting curve analysis is performed, wherein, during a process of temperature change, the double-stranded product formed by the probe (P) and the single-stranded amplification product (D1) will melt at a certain temperature, at this time, the probe (P) with the first detectable label and the second detectable label will be changed from the double-stranded state to the single-stranded state, so as to cause a change in the distance between the detectable labels, thereby generating a signal that may be detected by the instrument.

Due to the fact that a melting temperature of the double-stranded product formed by the probe (P) and the single-stranded amplification product (S1) is detected during melting curve analysis, it is possible to adjust the melting temperature of the double-stranded product formed by the probe (P) and the second single-stranded amplification product (D1) by adjusting a sequence and length of the primer signal detection region (h) of the reverse primer (R) or adjusting a sequence and length of the probe anchoring region A of the second forward primer (F0) to obtain the second single-stranded amplification product (D1) that is partially or completely complementary with the primer anchoring region (A′) of the probe (P) and is partially or completely complementary with the probe signal detection region (H) of the probe (P). For example, it is possible to increase or decrease the melting temperature of the double-stranded product formed by the probe (P) and the second single-stranded amplification product (D1) by adjusting a length or sequence of the primer signal detection region (h), or adjusting a position at which the complementary sequence (h′) of the primer signal detection is complementary with the probe signal detection region (H) of the probe (P).

When a multiplex PCR detection is performed, there may be multigroups of first forward primers (F) and reverse primers (R) targeting different target sequences, and different target sequences may be identified by means of fluorescence channels or melting temperatures; and when different target sequences are identified by means of fluorescence channels, different probes (P) and different second forward primers (F0) may be used for different target sequences, and when different target sequences are identified by means of melting temperatures, a same probe (P) may be shared by different target sequences.

In some specific embodiments, the first forward primer (F) has a length of 20-80 bases, wherein the target sequence binding region has a Tm value of 40° C.-80° C. and a GC content of 20%-80%; and the amplification product capturing region (B) has a length of 5-35 bases, a Tm value of 40° C.-80° C., and a GC content of 20%-80%.

In some specific embodiments, the second forward primer (F0) has a length of 20-80 bases, wherein the probe anchoring region (A) has a length of 5-65 bases, a Tm value of 40° C.-80° C., and a GC content of 30%-80%; and the amplification product capturing region (B) has a length of 5-35 bases, a Tm value of 40° C.-80° C., and a GC content of 20%-80%.

In some specific embodiments, the reverse primer (R) has a length of 20-80 bases, wherein the target sequence binding region has a Tm value of 40° C.-80° C., and a GC content of 20%-80%; and the primer signal detection region (h) has a length of 5-65 bases, a Tm value of 30° C.-80° C., and a GC content of 20%-80%; and the extension blocking region (M) has a length of 1-20 bp.

In some specific embodiments, the probe (P) has a length of 20-100 bases, wherein the primer anchoring region (A′) has a length of 5-65 bases, a Tm value of 40° C.-80° C., and a GC content of 30%-80%; more preferably, the primer anchoring region (A′) is designed at 5′ end of the probe (P); and the probe signal detection region (H) has a length of 5-65 bases, a Tm value of 30° C.-80° C., and a GC content of 20%-80%.

An annealing temperature of the probe and second forward primer is TmA, an annealing temperature of the probe and the complementary sequence of the reverse primer is TmH, an annealing temperature of the first forward primer and the target sequence is Tmb1, and an annealing temperature of the reverse primer and the target sequence is Tmb2. In some specific embodiments, the TmA<Tmb1 and TmA<Tmb2, and/or TmH<Tmb1 and TmH<Tmb2.

When there is no target to be detected, the primer anchoring region (A′) of the probe (P) is complementary with the probe anchoring region (A) of the second forward primer (F0), and the other part of the probe (P) is in a single-stranded state, and a curly shape is formed due to molecular flexibility; and at this time, a distance between the first detectable label and the second detectable label is relatively close, and the efficiency of fluorescence resonance energy transfer is relatively high. When a target to be detected is present, the first forward primer (F) and the reverse primer (R) specifically bind with a target sequence respectively and extend to produce a first double-stranded amplification product, wherein one single-stranded amplification product (S1) sequentially comprises the extension blocking region (M), the primer signal detection region (h), the target sequence, and a complementary sequence (B′) of the amplification product capturing region from 5′ end to 3′ end; and at this time, the amplification product capturing region (B) of the second forward primer (F0) may be complementary with the complementary sequence (B′) of the amplification product capturing region at 3′ end of the first single-stranded amplification product (S1) and extend to obtain a second double-stranded amplification product, wherein one second single-stranded amplification product (D1) sequentially comprises the probe anchoring region (A), the amplification product capturing region (B), the target sequence, a complementary sequence (h′) of the primer signal detection region (h), and a complementary sequence (M′) of the extension blocking region from 5′ end to 3′ end; the probe signal detection region (H) of the probe (P) is complementarily paired with the complementary sequence (h′) of the primer signal detection region of the second single-stranded amplification products (D1), the primer anchoring region (A′) of the probe (P) is complementarily paired with the probe anchoring region (A) at 5′ end of the second single-stranded amplification product (D1), and the complementary sequence (M′) of the extension blocking region at 3′ end of the single-stranded amplification product (D1) is not complementary with any part of the probe (P); and at this time, the probe (P) is changed from the single-stranded state to a double-stranded state, and the distance between the first detectable label and the second detectable label is changed, thereby generating a signal that may be detected by an instrument.

After the completion of PCR, when the melting curve analysis is performed, the double-stranded product formed by the probe (P) and the second single-stranded amplification product (D1) will melt at a certain temperature, such that the probe (P) with the first detectable label and the second detectable label becomes a single-stranded state, at this time, the distance between the first detectable label and the second detectable label is decreased, and the efficiency of fluorescence resonance energy transfer is increased, such that a fluorescence signal change is produced again, which may be used for melting curve analysis.

### Embodiment 3

In this embodiment, the probe primer composition comprises a probe, a first forward primer, a second forward primer, a first reverse primer, and a second reverse primer. The first forward primer (F) sequentially comprises a first amplification product capturing region (B) (a forward amplification product capturing region) and a target sequence binding region from 5′ end to 3′ end, wherein the target sequence binding region is a target specific sequence; the first amplification product capturing region (B) is a freely designed sequence that does not bind with any target sequence in a paired manner; and there are 0-20 bases spaced between the first amplification product capturing region (B) and the target sequence binding region.

The second forward primer (F0) comprises a probe anchoring region (A) and a first amplification product capturing region (B) from 5′ end to 3′ end.

The probe (P) is a freely designed sequence that does not bind with any target sequence in a paired manner, and comprises the primer anchoring region (A′) and a probe signal detection region (H); and the primer anchoring region (A′) is complementary with the probe anchoring region (A) of the second forward primer (F0).

In some specific embodiments, a first detectable label is labeled at a 1^{st}-95^{th} base position away from the primer anchoring region (A′), and a second detectable label is labeled at the base position spaced from the first detectable label by 5-25 bases. Both of the first detectable label and the second detectable label are located on the probe signal detection region (H), wherein the first detectable label may be a fluorescence reporter, and the second detectable label may be a quencher or other labeling groups that can produce a signal change with the first detectable label via fluorescence resonance energy transfer (FRET).

The first reverse primer (R) sequentially comprises a second amplification product capturing region (C) (a reverse amplification product capturing region), and a target sequence binding region from 5′ end to 3′ end, wherein the target sequence binding region is a target specific sequence; the second amplification product capturing region (C) is a freely designed sequence that does not bind with any target sequence in a paired manner; and there are 0-20 bases spaced between the second amplification product capturing region (C) and the target sequence binding region.

The second reverse primer (R0) sequentially comprises an extension blocking region (M), a primer signal detection region (h), and a second amplification product capturing region (C) from 5′ end to 3′ end.

During first PCR amplification, the first forward primer (F) and the first reverse primer (R) specifically bind with a target sequence respectively and extend to produce a first double-stranded amplification product, wherein one single-stranded amplification product (S1) sequentially comprises the first amplification product capturing region (B), the target sequence, and a complementary sequence (C′) of the second amplification product capturing region (C) from 5′ end to 3′ end, and wherein another single-stranded amplification product (S1′) sequentially comprises the second amplification product capturing region (C), the target sequence, and a complementary sequence (B′) of the first amplification product capturing region from 5′ end to 3′ end.

During second PCR amplification, the first double-stranded amplification product is taken as a template, and the first amplification product capturing region (B) of the second forward primer (F0) may be complementary with the complementary sequence (B′) of the first amplification product capturing region of the another first single-stranded amplification product (S1′) and extend; and the second amplification product capturing region (C) of the second reverse primer (R0) may be complementary with the complementary sequence (C′) of the second amplification product capturing region of the one first single-stranded amplification product (S1) and extend to obtain a second double-stranded amplification product, wherein one second single-stranded amplification product (D1) sequentially comprises the probe anchoring region (A), the first amplification product capturing region (B), the target sequence, the complementary sequence (C′) of the second amplification product capturing region, a complementary sequence (h′) of the primer signal detection region (h), and a complementary sequence (M′) of the extension blocking region from 5′ end to 3′ end.

After the second double-stranded amplification product is produced, the probe signal detection region (H) of the probe (P) is complementarily paired with the complementary sequence (h′) of the primer signal detection region of the one second single-stranded amplification products (D1), the primer anchoring region (A′) of the probe is complementarily paired with the probe anchoring region (A) at 5′ end of the second single-stranded amplification product (D1), and the complementary sequence (M′) of the extension blocking region at 3′ end of the single-stranded amplification product (S1) is not complementary with any part of the probe (P); and at this time, the probe (P) is changed from a single-stranded state to a double-stranded state, and a distance between the first detectable label and the second detectable label is changed, thereby generating a signal that may be detected by an instrument.

Due to the fact that a melting temperature of the double-stranded product formed by the probe (P) and the second single-stranded amplification product (D1) is detected during melting curve analysis, it is possible to adjust the melting temperature of the double-stranded product formed by the probe (P) and the second single-stranded amplification product (D1) by adjusting a sequence and length of the primer signal detection region (h) of the second reverse primer (R0) or adjusting a sequence and length of the probe anchoring region (A) of the second forward primer (F0) to obtain the second single-stranded amplification product (D1) that is partially complementary or completely complementary with the primer anchoring region (A′) of the probe (P) and is partially or completely complementary with the probe signal detection region (H) of the probe (P). For example, it is possible to increase or decrease the melting temperature of the double-stranded product formed by the probe (P) and the second single-stranded amplification product (D1) by adjusting a length or sequence of the primer signal detection region (h), or adjusting a position at which the complementary sequence (h′) of the primer signal detection region is complementary with the probe signal detection region (H) of the probe (P).

When a multiplex PCR detection is performed, there may be multigroups of first forward primers (F) and first reverse primers (R) targeting different target sequences, and different target sequences may be identified by means of fluorescence channels or melting temperatures; and when different target sequences are identified by means of fluorescence channels, different probes (P) as well as different second forward primers (F0) and second reverse primers (R0) may be used for different target sequences, and when different target sequences are identified by means of melting temperatures, a same probe (P), or a same second forward primer (F0) and a same second reverse primer (R0) may be shared by different target sequences.

In some specific embodiments, the first forward primer (F) has a length of 20-80 bases, wherein the target sequence binding region has a Tm value of 40° C.-80° C. and a GC content of 20%-80%; and the amplification product capturing region (B) has a length of 5-35 bases, a Tm value of 40° C.-80° C., and a GC content of 20%-80%.

In some specific embodiments, the second forward primer (F0) has a length of 20-80 bases, wherein the probe anchoring region (A) has a length of 5-65 bases, a Tm value of 40° C.-80° C., and a GC content of 30%-80%; and the amplification product capturing region (B) has a length of 5-35 bases, a Tm value of 40° C.-80° C., and a GC content of 20%-80%.

In some specific embodiments, the first reverse primer (R) has a length of 20-80 bases, wherein the target sequence binding region has a Tm value of 40° C.-80° C., and a GC content of 20%-80%; and the primer signal detection region (h) has a length of 5-65 bases, a Tm value of 30° C.-80° C., and a GC content of 20%-80%.

In some specific embodiments, the second reverse primer has a length of 20-80 bases, wherein the primer signal detection region has a length of 5-65 bases.

In some specific embodiments, the primer signal detection region of the second reverse primer has a Tm value of 30-80° C.

In some specific embodiments, the primer signal detection region of the second reverse primer has a GC content of 20-80%.

In some specific embodiments, the extension blocking region of the second reverse primer has a length of 1-20 bp.

In some specific embodiments, the probe (P) has a length of 20-100 bases, wherein the primer anchoring region (A′) has a length of 5-65 bases, a Tm value of 40° C.-80° C., and a GC content of 30%-80%; more preferably, the primer anchoring region (A′) is designed at 5′ end of the probe (P); and the probe signal detection region (H) has a length of 5-65 bases, a Tm value of 30° C.-80° C., and a GC content of 20%-80%.

An annealing temperature of the probe and second forward primer is TmA, an annealing temperature of the probe and the complementary sequence of the second reverse primer is TmH, an annealing temperature of the first forward primer and the target sequence is Tmb1, and an annealing temperature of the first reverse primer and the target sequence is Tmb2. In some specific embodiments, the TmA<Tmb1 and TmA<Tmb2, and/or TmH<Tmb1 and TmH<Tmb2.

When there is no target to be detected, the primer anchoring region (A′) of the probe (P) is complementary with the probe anchoring region (A) of the second forward primer (F0), and the other part of the probe (P) is in a single-stranded state, and a curly shape is formed due to molecular flexibility; and at this time, a distance between the first detectable label and the second detectable label is relatively close, and the efficiency of fluorescence resonance energy transfer is relatively high. When a target to be detected is present, the first forward primer (F) and the first reverse primer (R) specifically bind with a target sequence respectively and extend to produce a first double-stranded amplification product, and the second forward primer (F0) and the second reverse primer (R0) further amplify the first double-stranded amplification product to obtain a second double-stranded amplification product, wherein one second single-stranded amplification product (D1) sequentially comprises the probe anchoring region (A), the first amplification product capturing region (B), the target sequence, a complementary sequence (C′) of the second amplification product capturing region, a complementary sequence (h′) of the primer signal detection region, and a complementary sequence (M′) of the extension blocking region from 5′ end to 3′ end; the probe signal detection region (H) of the probe (P) is complementarily paired with the complementary sequence (h′) of the primer signal detection region of the second single-stranded amplification products (D1), the primer anchoring region (A′) of the probe (P) is complementarily paired with the probe anchoring region (A) at 5′ end of the one second single-stranded amplification product (D1), and the complementary sequence (M′) of the extension blocking region at 3′ end of the second single-stranded amplification product (D1) is not complementary with any part of the probe (P); and at this time, the probe (P) is changed from a single-stranded state to a double-stranded state, and the distance between the first detectable label and the second detectable label is changed, thereby generating a signal that may be detected by an instrument. After the completion of PCR, when the melting curve analysis is performed, the double-stranded product formed by the probe (P) and the second single-stranded amplification product (D1) will melt at a certain temperature, such that the probe (P) with the first detectable label and the second detectable label becomes a single-stranded state, at this time, the distance between the first detectable label and the second detectable label is decreased, and the efficiency of fluorescence resonance energy transfer is increased, such that a fluorescence signal change is produced again, which may be used for melting curve analysis.

Compared with the prior art, the advantages of the technical solution described in the present invention lie in:
Multiplex detection: when the method described in the present invention is used, both of a fluorescence channel analysis and melting temperature analysis in a single tube reaction may be simultaneously performed in two dimensions, that is, different targets may be detected by using a same fluorescence channel and by means of melting temperature characteristics; or, by using different fluorescent channels, the detection of target species may be achieved by multiplying the number of the fluorescent channels by the melting temperature characteristic, for example, when two melting temperature detection points are set in one fluorescence channel, if there are two fluorescence channels, 2×2 target sequences may be detected, thereby achieving a multiplex detection of more target sequences;
Low fluorescence background: for each fluorescence channel, only one probe is used, and then a differentiation may be achieved by the different melting temperatures of the amplification products, thereby greatly reducing the fluorescence background in the PCR reaction and improving reaction sensitivity;
Adjustable melting temperature: in the method described in the present invention, two ends of the probe are simultaneously combined with two ends of the amplification product and then the probe is stretched and light is emitted, and thus, by adjusting the number and sequence of bases in the probe anchoring region (A) and the complementary sequence (h′) of the primer signal detection region on the amplification product via primer design, it is possible to obtain different melting temperatures, such that different targets that share a same probe have different melting temperatures; and for example, by selecting sequences at different positions of the probe as signal detection region sequences, it is possible to obtain double-stranded products complementary with different parts of the probe, so as to differentiate the melting temperatures of different target products.
Good inclusiveness: compared with a Taqman hydrolysis probe method which requires three parts that are complementarily paired with the template, in the primer probe design method of the present invention, only two parts, namely the forward primer (F) and the reverse primer (R) are complementarily paired with the target sequence, such that when there are many high variation regions in the target sequence (such as viruses or bacterial genomes), the primer probe of the present invention has a better inclusiveness and a lower design difficulty;
Low cost: by means of the method of the present invention, a detection may be completed in a single tube reaction by only requiring a fluorescence quantitative PCR instrument, without the need for additional downstream instruments and without the need for chips or multiple reaction tubes for dividing reactions, thereby greatly reducing the cost of instruments and consumables, meanwhile, the number of fluorescence probes in the reagent is reduced, thereby greatly reducing the cost of the reagent, and allowing a large-scale promotion to be possible;
Single tube reaction: the method has low sample consumption, is particularly suitable for the detection of a rare sample, and can greatly increase a concentration of the sample added to the detection reaction, so as to improve detection sensitivity, and provide the possibility for a subsequent extraction-free one-tube detection;
Easy and simple operation: only a fluorescence quantitative PCR instrument is required for detection, without subsequent steps such as for capillary electrophoresis or nucleic acid hybridization, etc., meanwhile, the preparation is simple, and there is no need to manual operate after the instrument is enabled;
No easy to cause pollution: in the method described in the present invention, the instrument is completely closed after the sample addition is completed, without the need to open the cover for subsequent detection, thereby greatly reducing the possibility of polluting an experimental environment;
High sensitivity: the method described in the present invention has a very low requirement for the length of the target sequence, when the target type is a short fragment nucleic acid, such as a free nucleic acid, a higher sensitivity is achieved in detection for a shorter target sequence length; and
Wide applicable range: the present invention is suitable for nucleic acid detection of various sample types, including a serum sample, a plasma sample, a whole blood sample, a sputum sample, a swab sample, a lavage fluid sample, a fresh tissue sample, a formalin fixed paraffin embedded tissue (FFPE), and a target (such as bacteria, viruses, gene mutations), etc., such that clinical units, including primary hospitals, can quickly carry out a common target detection (such as detection of pathogenic microorganism infection), which may be assisted with other detection and definite diagnostic means, thereby quickly providing diagnostic evidence and medication schemes for clinical practice, reducing mental and economic burden of patients, and achieving an objective of precision medicine.

The preferred embodiments of the present invention will be described in the following, but the protection scope of the present invention is not limited to the following preferred embodiments. It should be noted that, several variations and improvements made by those skilled in the art based on the concept of the present invention belong to the protection scope of the present invention. The used reagents without specifying the manufacturer are all conventional products that may be commercially available.

### EXAMPLE

### Example 1: Detection of Respiratory Syncytial Virus (RSV) by Using a Probe Primer System

TABLE 1
Probe primer

| Name of | | | |
|---|---|---|---|
| primer | | Nucleotide | |
| probe | Number | sequence (5′-3′) | Label |
| P1 | SEQ ID | CCCTGGGAGCTCATGG | T14-CY5, |
| (probe) | NO: 1 | AGGTACTGGTCCTGTC | T26-BHQ1 |
| | | GACGCACG | 3′Spacer C3 |
| R1 | SEQ ID | TTATTTCATGGAGGTA | / |
| (Reverse | NO: 2 | CTGGTCCTGTCGAAAT | |
| primer) | | ATTGCAGATCCWACAC | |
| | | CTAACAA | |
| F1 | SEQ ID | GCTCCCAGGGTTTAAC | / |
| (Forward | NO: 3 | ATTAAGCAAGAAGAGG | |
| primer) | | AAAMGAAGAT | |

The forward primer F1 (SEQ ID NO: 3) is a specific primer designed for a target sequence of respiratory syncytial virus, and the F1 has a total length of 42 bp, wherein a 1^{st} to 29^{th} bases at 3′ end thereof serve as a target sequence binding region, and a 1^{st} to 10^{th} bases at 5′ end thereof serve as a probe anchoring region (A), which is complementary with a sequence of a 1^{st} to 10^{th} bases (i.e. a primer anchoring region (A′)) at 5′ end of the probe P1 (SEQ ID NO: 1).

The reverse primer R1 (SEQ ID NO: 2) is a specific primer designed for the target sequence of respiratory syncytial virus, and the R1 has a total length of 55 bp, wherein a 1^{st} to 24^{th} bases at 3′ end thereof serve as a target sequence binding region, a 1^{st} to 5^{th} bases at 5′ end thereof serve as an extension blocking region (M), and a 6^{th} to 28^{th} bases at 5′ end thereof serve as a primer signal detection region (h), which is identical to a sequence of a 8^{th} to 30^{th} bases at 3′ end of the probe P1 (SEQ ID NO: 1)

A reaction system comprises the above primer probe, a template, a DNA polymerase, a reverse transcriptase, and a reaction buffer, and is used for target specific amplification and fluorescence signal detection. The reaction system is involved in the following steps:
(1) Firstly, in a cycle of PCR amplification reaction, the forward primer (F1) and the reverse primer (R1) may specifically amplify a target nucleic acid sequence. After amplification, a double-stranded pre-amplification product will be obtained, wherein one single-stranded amplification product (S1) comprises the probe anchoring region (A), the target sequence, a complementary sequence (h′) of the primer signal detection region, and a complementary sequence (M′) of the extension blocking region from 5′ end to 3′ end; and the probe signal detection region (H) of the probe (P) is complementarily paired with the complementary sequence (h′) of the primer signal detection region of the single-stranded amplification product (S1), the primer anchoring region (A′) of the probe (P) is paired with the probe anchoring region (A) at 5′ end of the single-stranded amplification product (S1) in a reverse complementary manner, and the complementary sequence (M′) of the extension blocking region at 3′ end of the single-stranded pre-amplification product (S1) is not complementary with any part of the probe (P). At this time, the probe (P) is changed from a single-stranded state to a double-stranded state, and a distance between the first detectable label and the second detectable label is changed, thereby generating a signal that may be detected by the instrument.
(2) After the PCR amplification is completed, a melting curve analysis is performed, and the double-stranded product formed by the probe (P) and the single-stranded amplification product (S1) will melt at a certain temperature, at this time, the probe (P) with the first detectable label and the second detectable label will be changed to the single-stranded state, thereby generating a fluorescence signal change that may be detected by the instrument.

The reaction system for respiratory syncytial virus detection comprises the following components:

TABLE 2
Reaction system

| Reagents and components | | Concentration | |
|---|---|---|---|
| A system for | 2× PCR Reaction Buffer | 1× | |
| singleplex | DNA Polymerase | 2 | U |
| detection of | Reverse transcriptase | 5 | U |
| respiratory | Probe (P1) | 400 | nM |
| syncytial | Forward primer (F1) | 500 | nM |
| virus | Reverse primer (R1) | 100 | nM |

| | |
|---|---|
| Nucleic acid template | 0.1 ng-60 ng |
| Ultra-pure water | Adding to 20 μL |

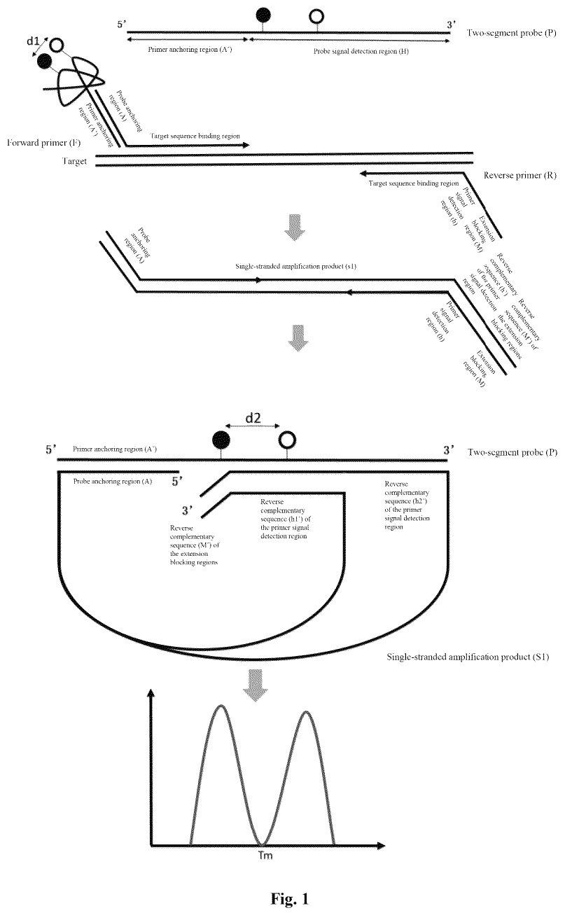

Among others, the nucleic acid template was derived from an in-vitro transcribed RNA sample. The reaction buffer and a primer probe premixed solution were mixed according to the reaction system described in Table 2, a nucleic acid was extracted from a sample to be detected and was added into the prepared reaction system, and then a PCR amplification and a melting curve analysis were performed. A pathogen type in the sample may be judged based on a characteristic Tm value of the target in a specific detection channel. A reaction principle was shown in FIG. 1.

The detection was performed by using an SLAN real-time fluorescence quantitative PCR instrument and reagent consumables from Shanghai Hongshi Medical Technology Co., Ltd. The 2×PCR reaction buffer comprises 3 mM MgCl₂, 30 mM Tris-HCl with a pH of 8.3, 0.5 mM dNTP, and 70 mM (NH₄)₂SO₄.

After a PCR tube was sealed by a cap, the sample was gently and uniformly mixed, and centrifuged for a short time and then left for 5 min at room temperature. The PCR tube was put in a handheld centrifuge again, centrifuged for a short time, and then transferred to a tray of the fluorescence quantitative PCR instrument. The procedure used was as follows: performing reverse transcription at 55° C. for 15 minutes; performing pre-denaturation at 95° C. for 2 minutes; performing denaturation at 94° C. for 10 seconds, and performing annealing and extension at 56° C. for 30 seconds, for a total of 45 cycles, without lighting; performing denaturation at 94° C. for 150 seconds, and performing incubation at a constant temperature of 40° C. for 300 seconds; and performing melting curve analysis at 40-85° C., with a heating rate of 0.05° C./s and with lighting.

### Data Analysis:

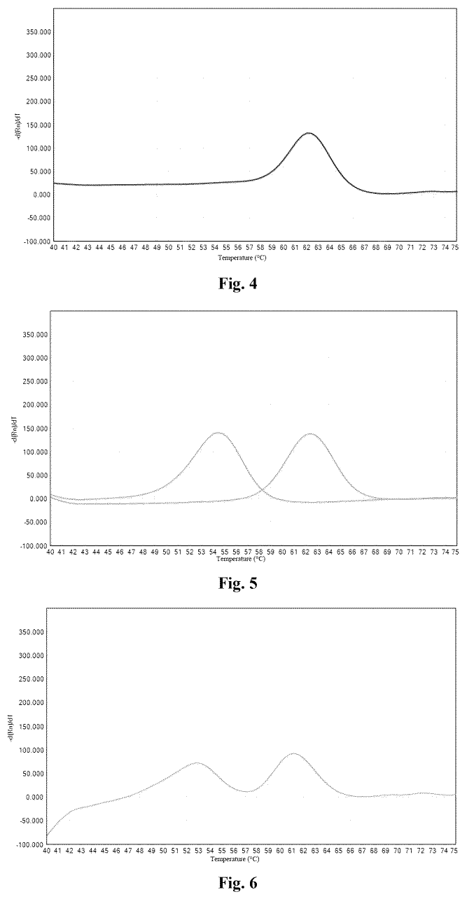

The detection results were subjected to data analysis by using an analysis software of the SLAN real-time fluorescence quantitative PCR instrument from Shanghai Hongshi Medical Technology Co., Ltd. The results were interpreted via Tm values of the melting curves. FIG. 4 is a melting curve of a singleplex PCR respiratory syncytial virus positive sample with the reverse primers (R) with different signal detection regions. FIG. 4 shows that a melting peak with a Tm value of 62.25° C. is formed in a PCR reaction well with addition of an RSV positive template in a CY5 channel.

### Example 2: Detection of Respiratory Syncytial Virus (RSV) and Influenza A Virus (IAV) by Using a Probe Primer System

TABLE 3
Probe primer

| Name of | | | |
|---|---|---|---|
| primer | | Nucleotide | |
| probe | Number | sequence (5′-3′) | Label |
| P2 | SEQ ID | CGCAAGTGAGGCGACT | T16-VIC |
| (Probe) | NO: 4 | ACGTGAGCGATGGACG | T27-BHQ2 |
| | | GAGGCAGGA | 3′ Spacer C3 |
| F3 | SEQ ID | GCCTCACTTGCGAAAA | / |
| (Forward | NO: 5 | AGCAACCAACAACATT | |
| primer) | | CATTG | |
| R3 | SEQ ID | TTTTTCTACGTGAGCG | / |
| (Reverse | NO: 6 | ATGGAAAGCAATTCAG | |
| primer) | | CATCACAGAC | |
| F4 | SEQ ID | GCCTCACTTGCGAAAA | / |
| (Forward | NO: 7 | TAATCAGGCACGAGAA | |
| primer) | | CAG | |
| R4 | SEQ ID | TTTTTGACTACGTGAG | / |
| (Reverse | NO: 8 | CGATGGACGGAGGAAA | |
| primer) | | AGACTAGCAACCTCCA | |
| | | TGGC | |

Both of the forward primer F3 (SEQ ID NO: 5) and the reverse primer R3 (SEQ ID NO: 6) are specific primers designed for a target sequence of respiratory syncytial virus. The F3 has a total length of 37 bp, wherein a 1^{st} to 22^{nd} bases at 3′ end thereof serve as a target sequence binding region, and a 1^{st} to 12^{th} bases at 5′ end thereof serve as a probe anchoring region (A), which is reverse complementary with a sequence of a 1^{st} to 12^{th} bases (i.e. a primer anchoring region (A′)) at 5′ end of the probe P2 (SEQ ID NO: 4). The R3 has a total length of 42 bp, wherein a 1^{st} to 19^{th} bases at 3′ end thereof serve as a target sequence binding region, a 1^{st} to 5^{th} bases at 5′ end thereof serve as an extension blocking region, and a 6^{th} to 20^{th} bases at 5′ end thereof serve as a primer signal detection region (h), which is identical to a sequence of a 13^{rd} to 27^{th} bases at 3′ end of the probe P2 (SEQ ID NO: 4).

Both of the forward primer F4 (SEQ ID NO: 7) and the reverse primer R4 (SEQ ID NO: 8) are specific primers designed for a target sequence of influenza A virus. The F4 has a total length of 35 bp, wherein a 1^{st} to 20^{th} bases at 3′ end thereof serve as a target sequence binding region, a 1^{st} to 12^{th} bases at 5′ end thereof serve as a probe anchoring region (A), which is reverse complementary with a sequence of a 1^{st} to 12^{th} bases (i.e. a primer anchoring region (A′)) at 5′ end of the probe P2 (SEQ ID NO: 4). The R4 has a total length of 52 bp, wherein a 1^{st} to 19^{th} bases at 3′ end thereof serve as a target sequence binding region, a 1^{st} to 5^{th} bases at 5′ end thereof serve as an extension blocking region (M), and a sequence a 6^{th} to 29^{th} bases at 5′ end thereof is identical to a sequence of a 6^{th} to 29^{th} bases at 3′ end of the probe P2 (SEQ ID NO: 4).

A reaction principle is shown in FIG. 1.

A reaction system for detection of respiratory syncytial virus (RSV) and influenza A virus (IAV) comprises the probe primer composition as shown in Table 3, a template, a DNA polymerase, a reverse transcriptase, and a reaction buffer, and is used for target specific amplification and fluorescence signal detection. Specifically, the reaction system comprises the following components:

TABLE 4
Reaction system

| Reagents and components | | Concentration | |
|---|---|---|---|
| A system for | 2× PCR Reaction Buffer | 1× | |
| detection of | DNA Polymerase | 2 | U |
| respiratory | Reverse transcriptase | 5 | U |
| syncytial | Forward Primer (F3) | 500 | nM |
| virus, and | Reverse primer (R3) | 100 | nM |
| influenza A | Probe (P2) | 400 | nM |
| virus | Forward primer (F4) | 500 | nM |
| | Reverse primer (R4) | 100 | nM |

| | |
|---|---|
| Nucleic acid template | 0.1 ng-60 ng |
| Ultra-pure water | Adding to 20 μL |

The nucleic acid template was derived from an in-vitro transcribed RNA sample. The reaction buffer and a primer probe premixed solution were mixed according to the reaction system described in Table 4, a nucleic acid was extracted from a sample to be detected and was added into the prepared reaction system, and then a PCR amplification and a melting curve analysis were performed. A pathogen type in the sample may be judged based on a characteristic Tm value of the target in a specific detection channel.

The detection was performed by using an SLAN real-time fluorescence quantitative PCR instrument and reagent consumables from Shanghai Hongshi Medical Technology Co., Ltd. The 2×PCR reaction buffer comprises 3 mM MgCl₂, 30 mM Tris-HCl with a pH of 8.3, 0.5 mM dNTP, 70 mM (NH₄)₂SO₄, etc.

In-vitro transcribed RNAs from individual target sequence were used as positive samples, and two target sequence templates of a same fluorescent channel were mixed for detection, to verify a detection ability for mixed virus infection. The ultra-pure water was used as a no template control (NTC).

### Reaction Preparation:

After the preparation of the samples was completed, the reaction system was prepared according to the ratios described in Table 4. An experimental principle of this example (i.e. a solution of the present invention) was shown in FIG. 1.

### PCR Amplification and Melting Curve Analysis:

After a PCR tube was sealed by a cap, the sample was gently and uniformly mixed, and centrifuged for a short time and then left for 5 min at room temperature. The PCR tube was put in a handheld centrifuge again, centrifuged for a short time, and then transferred to a tray of the fluorescence quantitative PCR instrument. The procedure used was as follows: performing reverse transcription at 55° C. for 15 minutes; performing pre-denaturation at 95° C. for 2 minutes; performing denaturation at 94° C. for 10 seconds, and performing annealing and extension at 56° C. for 30 seconds, for a total of 45 cycles, without lighting; performing denaturation at 94° C. for 150 seconds, and performing incubation at a constant temperature of 40° C. for 300 seconds; and performing melting curve analysis at 40-85° C., with a heating rate of 0.05° C./s and with lighting.

### Data Analysis:

The results were interpreted via Tm values of the melting curves. The detection results were subjected to data analysis by using an analysis software of the SLAN real-time fluorescence quantitative PCR instrument from Shanghai Hongshi Medical Technology Co., Ltd.

FIGS. 5-6 are melting curves of individual respiratory virus positive samples according to multiple PCR. FIG. 5 shows that dual melting peaks of RSV (52.93° C.) and IAV (61.17° C.) appear in a PCR reaction well with addition of an IAV positive template and an RSV positive template in a VIC channel, respectively. FIG. 6 shows that dual melting peaks of RSV (52.93° C.) and IAV (61.17° C.) simultaneously appear in a PCR reaction well with addition of both an IAV positive template and an RSV positive template in a VIC channel. It can be seen that, when the solution of the present invention is used to detect 2 pathogens, a good specificity is achieved, the melting curve has a single pattern, and the Tm values of two positive targets in a same channel may be clearly differentiated.

### Comparative Example: Detection of Respiratory Syncytial Virus (RSV) by Using a Primer Probe System in a Situation where a Primer Dimer is Extremely Easily Formed

TABLE 5
Probe primer

| Name of | | | |
|---|---|---|---|
| primer | | Nucleotide | |
| probe | Number | sequence (5′-3′) | Label |
| P5 | SEQ ID | CCCTGGGAGCTCATGG | T14-CY5, |
| (probe) | NO: 21 | AGGTACTGGTCCTGTC | T26-BHQ2 |
| | | GAC | 3′ Spacer C3 |
| P4 | SEQ ID | AGCGACGGTGCCGAGA | 3′BHQ1 |
| (probe) | NO: 10 | GGGT | 5′FAM |
| R5 | SEQ ID | TTATTTCATGGAGGTA | |
| (Reverse | NO: 11 | CTGGTCCTGTCGAAAT | |
| primer) | | ATTGCAGATACTCCAC | |
| | | CTCATCA | |
| F5 | SEQ ID | GCTCCCAGGGTTTAAC | |
| (Forward | NO: 12 | AGGCTGCAAGAAGAGG | |
| primer) | | ACTAGAAGAT | |
| R2 | SEQ ID | AGCGACGGTGCCGAGA | / |
| (Reverse | NO: 13 | GGGTAAATATTGCAGA | |
| primer) | | TACTCCACCTCATCA | |
| F2 | SEQ ID | AACAGGCTGCAAGAAG | / |
| (Forward | NO: 14 | AGGACTAGAAGAT | |
| primer) | | | |
| F2-1 | SEQ ID | TTTTTTTTTTGATGAG | / |
| (Forward | NO: 15 | GTGGAGTATCTGCAAT | |
| primer) | | A | |

In the above probe primer:
the forward primer F5 (SEQ ID NO: 12) is a specific primer designed for a target sequence of respiratory syncytial virus, and the F5 has a total length of 42 bp, wherein a 1^{st} to 29^{th} bases at 3′ end thereof serve as a target sequence binding region, and a 1^{st} to 10^{th} bases at 5′ end thereof serve as a probe anchoring region (A), which is reverse complementary with a sequence of a 1^{st} to 10^{th} bases (i.e. a primer anchoring region (A′)) at 5′ end of the probe P3 (SEQ ID NO: 9);
the reverse primer R5 (SEQ ID NO: 11) is a specific primer designed for the target sequence of respiratory syncytial virus, and the R5 has a total length of 55 bp, wherein a 1^{st} to 24^{th} bases at 3′ end thereof serve as a target sequence binding region, a 1^{st} to 5^{th} bases at 5′ end thereof serves as an extension blocking region (M), and a 6^{th} to 28^{th} bases at 5′ end thereof serves as a primer signal detection region (h), which is identical to a sequence of a 3^{rd} to 25^{th} bases at 3′ end of the probe P3 (SEQ ID NO: 9);
the forward primer F2 (SEQ ID NO: 14) is a specific primer designed for the target sequence of respiratory syncytial virus, and the F2 has a total length of 29 bp, and is identical to a 1^{st} to 29^{th} base at 3′ end of F5;
the reverse primer R2 (SEQ ID NO: 13) is a specific primer designed for the target sequence of respiratory syncytial virus, and the R2 has a total length of 47 bp, wherein a 1^{st} to 24^{th} bases at 3′ end thereof serve as a target sequence binding region and is identical to a sequence of a 1^{st} to 24^{th} bases at 3′ end of R5, and a sequence of a 1^{st} to 20^{th} bases at 5′ end thereof is completely identical to a base sequence of the probe P4 (SEQ ID NO: 10);
the forward primer F2-1 (SEQ ID NO: 15) is a specific dimer primer designed for the target sequence of respiratory syncytial virus, and the F2-1 has a total length of 33 bp, wherein a 1^{st} to 24^{th} bases at 3′ end thereof is complementary with a sequence of a 1^{st} to 24^{th} bases at 3′ end of the reverse primer R5 (SEQ ID NO: 11);
the probe P5 (SEQ ID NO: 21) has a nucleotide sequence consistent with that of the probe P3, except that the labeling group of probe P5 is different from that of the probe P3. The probe P5 is labeled thereon with a CY5 detectable label; and
the probe P4 (SEQ ID NO: 10) is a self-designed sequence that is not complementary with any part of the target sequence, and the P4 has a total length of 20 bp, and is identical to a sequence of a 1^{st} to 20^{th} bases at 5′ end of R2.

When a pathogen target is specifically amplified, the forward primer (F5) and the reverse primer (R5) amplify a target nucleic acid to obtain a double-stranded amplification product, wherein one single-stranded pre-amplification product (S1) sequentially comprises the probe anchoring region (A), the target sequence, a reverse complementary sequence (h′) of the primer signal detection region, and a reverse complementary sequence (M′) of the extension blocking region; and the probe signal detection region (H) of the probe (P5) is complementarily paired with the reverse complementary sequence (h′) of the primer signal detection region of the single-stranded pre-amplification product (S1), the primer anchoring region (A′) of the probe is paired with the probe anchoring region (A) at 5′ end of the single-stranded pre-amplification product (S1) in a reverse complementary manner, and the reverse complementary sequence (M′) of the extension blocking region at 3′ end of the single-stranded pre-amplification product (S1) is not complementary with any part of the probe (P5). At this time, the probe (P5) is changed from a single-stranded state to a double-stranded state, and a distance between the first detectable label and the second detectable label is changed, thereby generating a signal that may be detected by the instrument; and after the amplification is completed, a melting curve analysis is performed, wherein, during a process of temperature change, the double-stranded product formed by the probe (P5) and the single-stranded product will melt at a certain temperature, at this time, the probe (P5) with the first detectable label and the second detectable label will be changed to the single-stranded state, thereby generating a signal change that may be detected by the instrument

When the pathogen target is specifically amplified, the F2 (SEQ ID NO: 14) and the R2 (SEQ ID NO: 13) amplify the target nucleic acid to obtain a double-stranded pre-amplification product, wherein a 5′ end of one single-stranded pre-amplification product is reverse complementary with the probe P4 (SEQ ID NO: 10). At this time, a distance between the first detectable label and the second detectable label at two ends of the probe P4 (SEQ ID NO: 10) is increased due to a change from single strand to double strand, and the efficiency of fluorescence resonance energy transfer is decreased, such that a fluorescence signal change is produced and may be detected by an instrument. After the amplification is completed, a melting curve analysis is performed, and the double-stranded product formed with the probe P4 (SEQ ID NO: 10) will melt at a certain melting temperature, at this time, the probe P4 (SEQ ID NO: 10) with the first detectable label and the second detectable label will be changed to a single-stranded state, such that a fluorescence signal change is produced and may be detected by the instrument.

When there is no pathogen target in the sample to be detected, a double-stranded primer dimer is obtained by reverse complementary amplification of the forward primer F2-1 (SEQ ID NO: 15) and the reverse primer R5 (SEQ ID NO: 11), and since the forward primer F2-1 (SEQ ID NO: 15) does not contain a probe anchoring region (A) at 5′ end thereof, neither of the single strands of the obtained double-stranded primer dimer contains the probe anchoring region (A), then the reverse complementary sequence (h′) of the primer signal detection region cannot bind with the probe (P5) without the probe anchoring region (A). After amplification, a melting curve analysis is performed, and no positive signal is generated due to the absence of a double-stranded product formed with the probe P5 (SEQ ID NO: 21).

When there is no pathogen target in the sample to be detected, a double-stranded primer dimer is obtained by reverse complementary amplification of the forward primer F2-1 (SEQ ID NO: 15) and the reverse primer R5 (SEQ ID NO: 13), wherein one single-stranded product may be reverse complementary with the probe P4 (SEQ ID NO: 10), such that a distance between the first detectable label and the second detectable label at two ends of the probe P4 (SEQ ID NO: 10) is increased due to a change from single strand to double strand, and the efficiency of fluorescence resonance energy transfer is decreased, and thus a fluorescence signal change is produced and may be detected by an instrument, thereby generating a false positive signal. After the amplification is completed, a melting curve analysis is performed, and the double-stranded product formed with the probe P4 (SEQ ID NO: 10) will melt at a certain melting temperature, at this time, the probe P4 (SEQ ID NO: 10) with the first detectable label and the second detectable label will be changed to a single-stranded state, such that a fluorescence signal change is produced and may be detected by the instrument, thereby generating a false positive signal.

A reaction system for detection of respiratory syncytial virus (RSV) in a situation where a primer dimer is extremely easily formed, comprises the primer probe as shown in above Table 5, a template, a DNA polymerase, a reverse transcriptase, and a reaction buffer, and is used for target specific amplification and fluorescence signal detection. The reaction system is involved in the following steps:

In a cycle of PCR amplification reaction, the forward primer (E5) and the reverse primer (R5) may specifically amplify a target nucleic acid sequence. After amplification, a double-stranded amplification product will be obtained, wherein one single-stranded amplification product (S1) comprises the probe anchoring region (A), the target sequence, a reverse complementary sequence (h′) of the primer signal detection region, and a reverse complementary sequence (M′) of the extension blocking region from 5′ end to 3′ end; the probe signal detection region (H) of the probe (P5) is complementarily paired with the reverse complementary sequence (h′) of the primer signal detection region of the single-stranded amplification product (S1), the primer anchoring region (A′) of the probe (P5) is paired with the probe anchoring region (A′) at 5′ end of the single-stranded amplification product (S1) in a reverse complementary manner, and the reverse complementary sequence (M′) of the extension blocking region at 3′ end of the single-stranded amplification product (S1) is not complementary with any part of the probe (P5). At this time, the probe (P5) is changed from a single-stranded state to a double-stranded state, and a distance between the first detectable label and the second detectable label is changed, thereby generating a signal that may be detected by the instrument. After the PCR amplification is completed, a melting curve analysis is performed, and the double-stranded product formed by the probe (P5) and the single-stranded amplification product (S1) will melt at a certain temperature, at this time, the probe (P5) with the first detectable label and the second detectable label will be changed to the single-stranded state, thereby generating a signal change that may be detected by the instrument.

In a cycle of PCR amplification reaction, a double-stranded primer dimer is obtained by reverse complementary amplification of the forward primer (F2-1) and the reverse primer (R5), and since the double-stranded primer dimer does not contain the probe anchoring region (A), then the reverse complementary sequence (h′) of the primer signal detection region cannot bind with the probe (P5) without the probe anchoring region (A). After amplification, a melting curve analysis is performed, and no positive signal is generated due to the absence of a double-stranded product formed with the probe (P5).

In a cycle of PCR amplification reaction, the forward primer (F2) and the reverse primer (R2) may amplify a target nucleic acid sequence. After amplification, one single-stranded amplification product may be reversely complementary with the probe (P4), such that a distance between the fluorescence group and the quencher group at two ends of the probe (P4) is changed, thereby generating a fluorescence signal change that may be detected by the instrument. After the PCR amplification is completed, a melting curve analysis is performed, and the double-stranded product formed with the probe (P4) will melt at a certain melting temperature, at this time, the probe (P4) with the first detectable label and the second detectable label will be changed to a single-stranded state, such that a fluorescence signal change is produced and may be detected by the instrument.

In a cycle of PCR amplification reaction, a double-stranded primer dimer is obtained by reverse complementary amplification of the forward primer (F2-1) and the reverse primer (R2). After amplification, one single-stranded amplification product may be reverse complementary with the probe (P4), such that a distance between the fluorescence group and the quencher group at two ends of the probe (P4) is changed, thereby generating a fluorescence signal change that may be detected by the instrument, and obtaining a false positive signal. After the PCR amplification is completed, a melting curve analysis is performed, and the double-stranded product formed with the probe (P4) will melt at a certain melting temperature, at this time, the probe (P4) with the first detectable label and the second detectable label will be changed to a single-stranded state, such that a fluorescence signal change is produced and may be detected by the instrument, and a false positive signal is obtained.

The system for detection of respiratory syncytial virus (RSV) in a situation where a primer dimer is extremely easily formed, comprises the following components:

TABLE 6
Reaction system in Comparative Example

| Reagents and components | | Concentration | |
|---|---|---|---|
| Reaction | 2× PCR Reaction Buffer | 1× | |
| system for | DNA Polymerase | 2 | U |
| singleplex | Reverse transcriptase | 5 | U |
| detection of | Forward primer (F5) | 500 | nM |
| respiratory | Reverse primer (R5) | 100 | nM |
| syncytial | Probe (P5) | 400 | nM |
| virus (P5 | Nucleic acid template | 0.1 ng-60 ng | |
| probe system) | Ultra-pure water | Adding to 20 μL | |
| Reaction | 2× PCR Reaction Buffer | 1× | |
| system for | DNA Polymerase | 2 | U |
| singleplex | Reverse transcriptase | 5 | U |
| detection of | Forward primer (F2-1) | 100 | nM |
| respiratory | Reverse primer (R5) | 100 | nM |
| syncytial | Probe (P5) | 400 | nM |
| virus (P5 | Nucleic acid template | 0.1 ng-60 ng | |
| probe primer | Ultra-pure water | Adding to 20 μL | |
| dimer system) | | | |
| Reaction | 2× PCR Reaction Buffer | 1× | |
| system for | DNA Polymerase | 2 | U |
| singleplex | Reverse transcriptase | 5 | U |
| detection of | Forward primer (F2) | 500 | nM |
| respiratory | Reverse primer (R2) | 100 | nM |
| syncytial | Probe (P4) | 400 | nM |
| virus (P4 | Nucleic acid template | 0.1 ng-60 ng | |
| probe system) | Ultra-pure water | Adding to 20 μL | |
| Reaction | 2× PCR Reaction Buffer | 1× | |
| system for | DNA Polymerase | 2 | U |
| singleplex | Reverse transcriptase | 5 | U |
| detection of | Forward primer (F2-1) | 100 | nM |
| respiratory | Reverse primer (R2) | 100 | nM |
| syncytial | Probe (P4) | 400 | nM |

| | | |
|---|---|---|
| virus (P4 | Nucleic acid template | 0.1 ng-60 ng |
| probe primer | Ultra-pure water | Adding to 20 μL |
| dimer system) | | |

The nucleic acid template was derived from an in-vitro transcribed RNA sample. The reaction buffer and a primer probe premixed solution were mixed according to the 4 reaction systems described in Table 6 respectively, a nucleic acid was extracted from a sample to be detected and was added into each of the prepared reaction systems, and then a PCR amplification and a melting curve analysis were performed. A pathogen type in the sample may be judged based on a characteristic Tm value of each target in a specific detection channel.

The detection was performed by using a SLAN real-time fluorescence quantitative PCR instrument and reagent consumables from Shanghai Hongshi Medical Technology Co., Ltd. The 2×PCR reaction buffer comprises 3 mM MgCl₂, 30 mM Tris-HCl with a pH of 8.3, 0.5 mM dNTP, and 70 mM (NH₄)₂SO₄.

In-vitro transcribed RNAs from target sequences of respiratory syncytial virus were used as positive samples, and the reverse primers (R) with different signal detection region sequences were used to separately detect the target sequences of respiratory syncytial virus respectively, to verify a detection ability for single virus infection. The pure water was used as a no template control (NTC).

### Reaction Preparation:

After the preparation of the samples was completed, the reaction systems were prepared according to the ratios described in Table 6.

### PCR Amplification and Melting Curve Analysis:

After a PCR tube was sealed by a cap, the sample was gently and uniformly mixed, and centrifuged for a short time and then left for 5 min at room temperature. The PCR tube was put in a handheld centrifuge again, centrifuged for a short time, and then transferred to a tray of the fluorescence quantitative PCR instrument. The procedure used was as follows: performing reverse transcription at 55° C. for 15 minutes; performing pre-denaturation at 95° C. for 3 minutes; performing denaturation at 94° C. for 10 seconds, and performing annealing and extension at 56° C. for 30 seconds, for a total of 45 cycles, without lighting; performing denaturation at 94° C. for 150 seconds, and performing incubation at a constant temperature of 40° C. for 300 seconds; and performing melting curve analysis at 40-85° C., with a heating rate of 0.05° C./s and with lighting.

### Data Analysis:

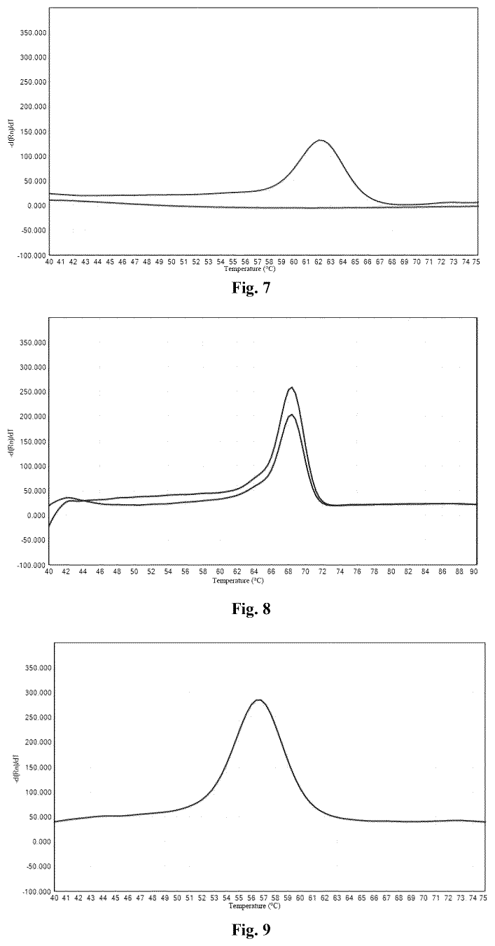

The results were interpreted via Tm values of the melting curves. A data analysis was performed by using an analysis software of the SLAN real-time fluorescence quantitative PCR instrument from Shanghai Hongshi Medical Technology Co., Ltd. FIG. 7 shows melting curves of single PCR respiratory syncytial virus positive samples and a primer dimer that are amplified by using probe P5. FIG. 8 shows melting curves of single PCR respiratory syncytial virus positive samples and a primer dimer that are amplified by using probe P4.

FIG. 7 shows that a melting peak with a Tm value of 62.38° C. is formed in a PCR reaction well with addition of an RSV positive template in a CY5 channel when the probe P5 is used, while the primer dimer does not produce a melting peak; and

FIG. 8 shows that a melting peak with a Tm value of 68.48° C. and a primer dimer melting peak with a Tm value of 68.52° C. are formed in a PCR reaction well with addition of an RSV positive template in an FAM channel when the probe P4 is used.

Thus it can be seen that, when the probe primer composition of the present invention is used, false positives generated by primer dimers may be effectively avoided.

### Example 3: Detection of Respiratory Syncytial Virus (RSV) by Using a Probe Primer System

TABLE 7
Probe primer

| Name of | | | |
|---|---|---|---|
| primer probe | Number | Nucleotide sequence (5′-3′) | Label |
| P3 (Probe) | SEQ ID NO:9 | CCCTGGGAGCTCATGGAGGTACTGGT | T14-FAM, |
| | | CCTGTCGAC | T26-BHQ1 |
| | | | 3′ Spacer C3 |
| R6 (Reverse | SEQ ID NO:16 | 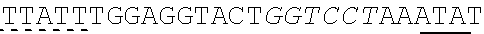 | / |
| primer) | | TGCAGATACTCCACCTCATCA | |
| F6 (First | SEQ ID NO:17 | GACTGCAGTCAATTTAACAGGCTGCA | / |
| forward | | AGAAGAGGACTAGAAGAT | |
| primer) | | | |
| F6-1 (Second forward | SEQ ID NO:18 | 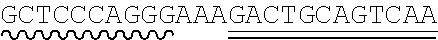 | / |
| primer) | | | |

### in the Above Probe Primer:

the first forward primer F6 (SEQ ID NO: 17) is a specific primer designed for a target sequence of respiratory syncytial virus, and the F6 has a total length of 44 bp, wherein a 1^{st} to 29^{th} bases at 3′ end thereof serve as a target sequence binding region, and a 1^{st} to 12^{th} bases at 5′ end thereof serve as an amplification product capturing region (B);
the reverse primer R6 (SEQ ID NO: 16) is a specific primer designed for the target sequence of respiratory syncytial virus, and the R6 has a total length of 48 bp, wherein a 1^{st} to 24^{th} bases at 3′ end thereof serve as a target sequence binding region, a 1^{st} to 5^{th} bases at 5′ end thereof serve as an extension blocking region (M), and a 6^{th} to 21^{st} bases at 5′ end thereof serve as a primer signal detection region (h), which is identical to a sequence of a 7^{th} to 22^{th} bases at 3′ end of the probe P3 (SEQ ID NO: 9);
and the second forward primer F6-1 (SEQ ID NO: 18) has a total length of 25 bp, wherein a 1^{st} to 12^{th} bases at 3′ end thereof serve as an amplification product capturing region (B), and a 1^{st} to 10^{th} bases at 5′ end thereof serve as a probe anchoring region (A), which is reverse complementary with a sequence of a 1^{st} to 10^{th} bases (i.e. a primer anchoring region (A′)) at 5′ end of the probe P3 (SEQ ID NO: 9). A reaction principle is shown in FIG. 2.

A reaction system for detection of respiratory syncytial virus (RSV) comprises the above probe primer composition described in Table 7, a template, a DNA polymerase, a reverse transcriptase, and a reaction buffer, and is used for target specific amplification and fluorescence signal detection. Specifically, the reaction system comprises the following components:

TABLE 8
Reaction system in this Example

| | Reagents and components | Concentration | |
|---|---|---|---|
| System for | 2× PCR Reaction Buffer | 1× | |
| singleplex | DNA Polymerase | 2 | U |
| detection of | Reverse transcriptase | 5 | U |
| respiratory | Probe (P3) | 400 | nM |
| syncytial | First forward primer (F6) | 100 | nM |
| virus | Reverse primer (R6) | 45 | nM |
| | Second forward primer (F6-1) | 200 | nM |

| | |
|---|---|
| Nucleic acid template | 0.1 ng-60 ng |
| Ultra-pure water | Adding to 20 μL |

Among others, according to a specific embodiment of the present invention, the nucleic acid template was derived from an in-vitro transcribed RNA sample. The reaction buffer and a primer probe premixed solution were mixed according to the reaction system described in Table 8, a nucleic acid was extracted from a sample to be detected and was added into the prepared reaction system, and then a PCR amplification and a melting curve analysis were performed. A pathogen type in the sample may be judged based on a characteristic Tm value of the target in a specific detection channel.

The detection was performed by using an SLAN real-time fluorescence quantitative PCR instrument and reagent consumables from Shanghai Hongshi Medical Technology Co., Ltd. The 2×PCR reaction buffer comprises 3 mM MgCl₂, 30 mM Tris-HCl with a pH of 8.3, 0.5 mM dNTP, and 70 mM (NH₄)₂SO₄.

An in-vitro transcribed RNA from the target sequence of respiratory syncytial virus was used as a positive sample, and the reverse primers (R) with different signal detection region sequences were used to separately detect the target sequence of respiratory syncytial virus respectively, to verify a detection ability for single virus infection. The pure water was used as a no template control (NTC).

### Reaction Preparation:

After the preparation of the samples was completed, the reaction system was prepared according to the ratios described in Table 8. The reaction principle was shown in FIG. 2

### PCR Amplification and Melting Curve Analysis:

After a PCR tube was sealed by a cap, the sample was gently and uniformly mixed, and centrifuged for a short time and then left for 5 min at room temperature. The PCR tube was put in a handheld centrifuge again, centrifuged for a short time, and then transferred to a tray of the fluorescence quantitative PCR instrument. The procedure used was as follows: performing reverse transcription at 55° C. for 15 minutes; performing pre-denaturation at 95° C. for 2 minutes; performing denaturation at 94° C. for 10 seconds, and performing annealing and extension at 56° C. for 30 seconds, for a total of 45 cycles, without lighting; performing denaturation at 94° C. for 150 seconds, and performing incubation at a constant temperature of 40° C. for 300 seconds; and performing melting curve analysis at 40-95° C., with a heating rate of 0.05° C./s and with lighting.

### Data Analysis:

The results were interpreted via a Tm value of the melting curve. A data analysis was performed by using an analysis software of the SLAN real-time fluorescence quantitative PCR instrument from Shanghai Hongshi Medical Technology Co., Ltd.

FIG. 9 shows that a melting peak with a Tm value of 56.62° C. is formed in a PCR reaction well with addition of an RSV positive template in an EAM channel in Example 3.

### Example 4: Detection of Respiratory Syncytial Virus (RSV) by Using a Probe Primer System

TABLE 9
Probe primer composition

| Name of | | | |
|---|---|---|---|
| primer | | Nucleotide | |
| probe | Number | sequence (5′-3′) | Label |
| P3 | SEQ ID | CCCTGGGAGCTCATGG | T14-FAM, |
| (Probe) | NO: 9 | AGGTACTGGTCCTGTC | T26-BHQ1 |
| | | GAC | 3′ Spacer C3 |
| R7 | SEQ ID | CGCATTACAAGTTAAA | / |
| (First | NO: 19 | TATTGCAGATACTCCA | |
| reverse | | CCTCATCA | |
| primer) | | | |
| F6 | SEQ ID | GACTGCAGTCAATTTA | / |
| (First | NO: 17 | ACAGGCTGCAAGAAGA | |
| forward | | GGACTAGAAGAT | |
| primer) | | | |
| F6-1 | SEQ ID | GCTCCCAGGGAAAGAC | / |
| (Second | NO: 18 | TGCAGTCAA | |
| forward | | | |
| primer) | | | |
| R7-1 | SEQ ID | TTATTTGGAGGTACTG | / |
| (Second | NO: 20 | GTCCTAAACGCATTAC | |
| reverse | | AAGTT | |
| primer) | | | |

### in the Above Primer Probe:

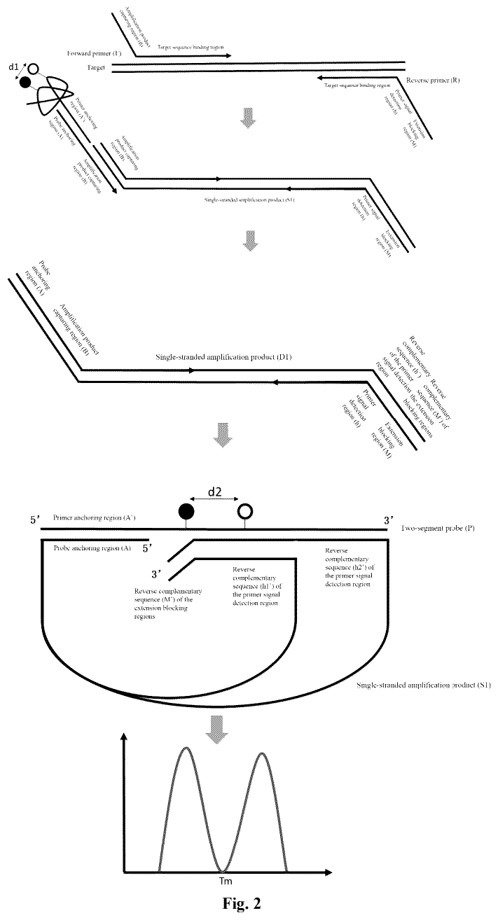

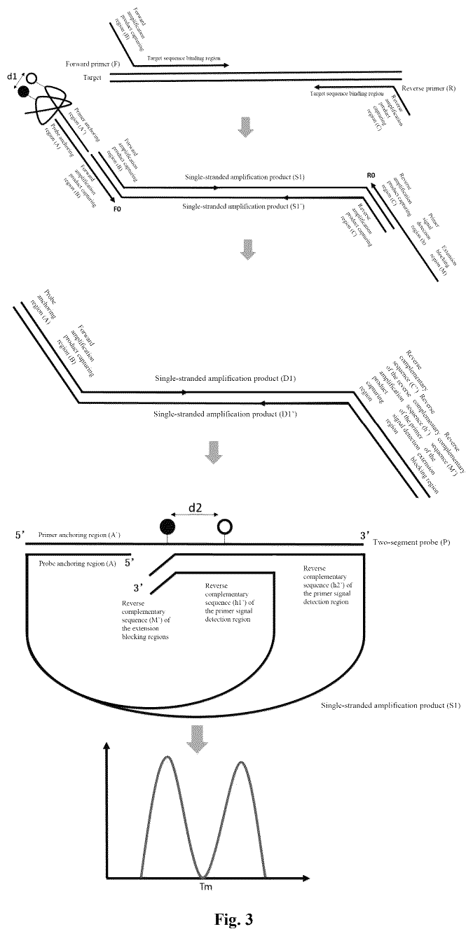

the first forward primer F6 (SEQ ID NO: 17) is a specific primer designed for a target sequence of respiratory syncytial virus, and the F6 has a total length of 44 bp, wherein a 1^{st} to 29^{th} bases at 3′ end thereof serve as a target sequence binding region, and a 1^{st} to 12^{th} bases at 5′ end thereof serve as an amplification product capturing region (B);
the first reverse primer R7 (SEQ ID NO: 19) is a specific primer designed for the target sequence of respiratory syncytial virus, and the R7 has a total length of 40 bp, wherein a 1^{st} to 24^{th} bases at 3′ end thereof serve as a target sequence binding region, and a 1^{st} to 13^{th} bases at 5′ end thereof serve as a second amplification product capturing region (C);
the second forward primer F6-1 (SEQ ID NO: 18) has a total length of 25 bp, wherein a 1^{st} to 12^{th} bases at 3′ end thereof serve as an amplification product capturing region (B), and a 1^{st} to 10^{th} bases at 5′ end thereof serve as a probe anchoring region (A), which is reverse complementary with a sequence of a 1^{st} to 10^{th} bases (i.e. a primer anchoring region (A′)) at 5′ end of the probe P3 (SEQ ID NO: 9); and
the second reverse primer R7-1 (SEQ ID NO: 20) has a total length of 37 bp, wherein a 1^{st} to 5^{th} bases at 5′ end thereof serve as extension blocking region (M), a 6^{th} to 21^{st} bases at 5′ end thereof serve as a primer signal detection region (h), and a 1^{st} to 13^{th} bases at 3′ end thereof serve as a second amplification product capturing region (C). A reaction principle is shown in FIG. 3.

Specifically, a reaction system for detection of respiratory syncytial virus comprises the following components:

TABLE 10
Reaction system in this Example

| | |
|---|---|
| Reagents and components | Concentration |

| System for | 2× PCR Reaction Buffer | 1× | |
|---|---|---|---|
| singleplex | DNA Polymerase | 2 | U |
| detection of | Reverse transcriptase | 5 | U |
| respiratory | Probe (P3) | 400 | nM |
| syncytial | First forward primer (F6) | 100 | nM |
| virus | First reverse primer (R7) | 45 | nM |
| | Second forward primer (F6-1) | 250 | nM |
| | Second reverse primer (R7-1) | 100 | nM |

| | |
|---|---|
| Nucleic acid template | 0.1 ng-60 ng |
| Ultra-pure water | Adding to 20 μL |

The nucleic acid template was derived from an in-vitro transcribed RNA sample. The reaction buffer and a primer probe premixed solution were mixed according to the reaction system described in Table 10, a nucleic acid was extracted from a sample to be detected and was added into the prepared reaction system, and then a PCR amplification and a melting curve analysis were performed. A pathogen type in the sample may be judged based on a characteristic Tm value of the target in a specific detection channel.

The detection was performed by using an SLAN real-time fluorescence quantitative PCR instrument and reagent consumables from Shanghai Hongshi Medical Technology Co., Ltd. The 2×PCR reaction buffer comprises 3 mM MgCl₂, 30 mM Tris-HCl with a pH of 8.3, 0.5 mM dNTP, and 70 mM (NH₄)₂SO₄.

Sample preparation: an in-vitro transcribed RNA from the target sequence of respiratory syncytial virus was used as a positive sample, and the reverse primers (R) with different signal detection region sequences were used to separately detect the target sequence of respiratory syncytial virus respectively, to verify a detection ability for single virus infection. The pure water was used as a no template control (NTC).

### Reaction Preparation:

After the preparation of the samples was completed, the reaction system was prepared according to the ratios described in Table 10. The reaction principle was shown in FIG. 3

### PCR Amplification and Melting Curve Analysis:

After a PCR tube was sealed by a cap, the sample was gently and uniformly mixed, and centrifuged for a short time and then left for 5 min at room temperature. The PCR tube was put in a handheld centrifuge again, centrifuged for a short time, and then transferred to a tray of the fluorescence quantitative PCR instrument. The procedure used was as follows: performing reverse transcription at 55° C. for 15 minutes; performing pre-denaturation at 95° C. for 2 minutes; performing denaturation at 94° C. for 10 seconds, and performing annealing and extension at 56° C. for 30 seconds, for a total of 45 cycles, without lighting; performing denaturation at 94° C. for 150 seconds, and performing incubation at a constant temperature of 40° C. for 300 seconds; and performing melting curve analysis at 40-85° C., with a heating rate of 0.05° C./s and with lighting.

### Data Analysis:

The results were interpreted via a Tm value of the melting curve. A data analysis was performed by using an analysis software of the SLAN real-time fluorescence quantitative PCR instrument from Shanghai Hongshi Medical Technology Co., Ltd.

FIG. 10 shows that a melting peak with a Tm value of 56.36° C. is formed in a PCR reaction well with addition of an RSV positive template in an FAM channel in Example 4.

### Example 5: Singleplex Detection of Non-Small Cell Lung Cancer EGFR Exon 20 H773_V774insH Mutation by Using a Primer Probe System

TABLE 11
Primer probe

| Name of | | | |
|---|---|---|---|
| primer | | Nucleotide | |
| probe | Number | sequence (5′-3′) | Label |
| P6 | SEQ ID | CCACGTCTCAACTATG | C1-FAM, |
| | NO: 22 | ACACTCCTCCGACCAT | T15-BHQ1 |
| | | CCGCTTCCTCCA | 3′ Spacer C3 |
| R8 | SEQ ID | CCACGTCTCAACTATA | / |
| | NO: 23 | AAAGTCTGAACGACCT | |
| | | ACACTCC | |
| F8 | SEQ ID | TGGAGGAAGCGGATGT | / |
| | NO: 24 | TTCCCAGCAGCTGACA | |
| | | CA | |

### in the Above Probe Primer Composition:

the forward primer F8 (SEQ ID NO: 24) is a specific primer designed for a target sequence of non-small cell lung cancer EGFR exon 20 H773_V774insH mutation, and the F8 has a total length of 34 bp, wherein a 1^{st} to 16^{th} bases at 3′ end thereof serve as a target sequence binding region, and a 1^{st} to 15^{th} bases at 5′ end thereof serve as a probe anchoring region (A), which is reverse complementary with a sequence of a 1^{st} to 15^{th} bases (i.e. a primer anchoring region (A′)) at 3′ end of the probe P6 (SEQ ID NO: 22); and
the reverse primer R8 (SEQ ID NO: 23) is a specific primer designed for the target sequence of non-small cell lung cancer EGFR exon 20 H773_V774insH mutation, and the R8 has a total length of 39 bp, wherein a 1^{st} to 21^{st} bases at 3′ end thereof serve as a target sequence binding region, a 1^{st} to 15^{th} bases at 5′ end thereof serve as a primer signal detection region (h), which is identical to a sequence of a 1^{st} to 15^{th} bases at 5′ end of the probe P6 (SEQ ID NO: 22)

A reaction system comprises the above primer probe, a template, a DNA polymerase, a reverse transcriptase, and a reaction buffer, and is used for target specific amplification and fluorescence signal detection. Specifically, the reaction system is involved in the following steps:
(1) Firstly, in a cycle of PCR amplification reaction, the forward primer (F8) and the reverse primer (R8) may specifically amplify a target nucleic acid sequence. After amplification, a double-stranded amplification product will be obtained, wherein one single-stranded amplification product (S1) comprises the probe anchoring region (A), the target sequence, and a reverse complementary sequence (h′) of the primer signal detection region from 5′ end to 3′ end; and the probe signal detection region (H) of the probe (P6) is complementarily paired with the reverse complementary sequence (h′) of the primer signal detection region of the single-stranded amplification product (S1), and the primer anchoring region (A′) of the probe (P6) is paired with the probe anchoring region (A) at 5′ end of the single-stranded amplification product (S1) in a reverse complementary manner. At this time, a distance between the first detectable label and the second detectable label of the probe (P) is changed, thereby generating a signal that may be detected by the instrument.
(2) After the PCR amplification is completed, a melting curve analysis is performed, and the double-stranded product formed by the probe (P6) and the single-stranded amplification product (S1) will melt at a certain temperature, at this time, the probe (P6) with the first detectable label and the second detectable label will be changed to the single-stranded state, thereby generating a fluorescence signal change that may be detected by the instrument.

The reaction system comprises the following components:

TABLE 12
Reaction system in this Example

| | |
|---|---|
| Reagents and components | Concentration |

| System for | 2× PCR Reaction Buffer | 1× | |
|---|---|---|---|
| singleplex | DNA Polymerase | 2 | U |
| detection of | Probe (P6) | 400 | nM |
| non-small cell | Forward primer (F8) | 500 | nM |
| lung cancer | Reverse primer (R8) | 100 | nM |
| EGFR exon 20 | Nucleic acid template | 30 | copies |

| | | |
|---|---|---|
| H773_V774insH | Ultra-pure water | Adding to 20 μL |
| mutation | | |

A non-small cell lung cancer EGFR exon 20 H773_V774insH mutation was used as a positive sample, and the reverse primer (R) with a signal detection region sequence was used to separately detect the target sequence of non-small cell lung cancer EGFR exon 20 H773_V774insH mutation, to verify a detection ability for single mutation. The pure water was used as a no template control (NTC). A reaction principle was shown in FIG. 11.

The reaction buffer and a primer probe premixed solution were mixed according to the reaction system described in Table 12, a nucleic acid was extracted from a sample to be detected by a suitable method and was added into the prepared reaction system, and then a PCR amplification and a melting curve analysis were performed. An EGFR mutation type in the sample may be judged based on a characteristic Tm value of the target in a specific detection channel.

The detection was performed by using an SLAN real-time fluorescence quantitative PCR instrument and reagent consumables from Shanghai Hongshi Medical Technology Co., Ltd. The 2×PCR reaction buffer comprises 3 mM MgCl₂, 30 mM Tris-HCl with a pH of 8.3, 0.5 mM dNTP, 70 mM (NH₄)₂SO₄, etc.

### PCR Amplification and Melting Curve Analysis:

After a PCR tube was sealed by a cap, the sample was gently and uniformly mixed, and centrifuged for a short time and then left for 5 min at room temperature. The PCR tube was put in a handheld centrifuge again, centrifuged for a short time, and then transferred to a tray of the fluorescence quantitative PCR instrument. The procedure used was as follows: performing pre-denaturation at 95° C. for 3 minutes; performing denaturation at 94° C. for 10 seconds, and performing annealing and extension at 56° C. for 30 seconds, for a total of 45 cycles; and performing melting curve analysis at 40-85° C., with a heating rate of 0.05° C./s and with lighting.

### Data Analysis:

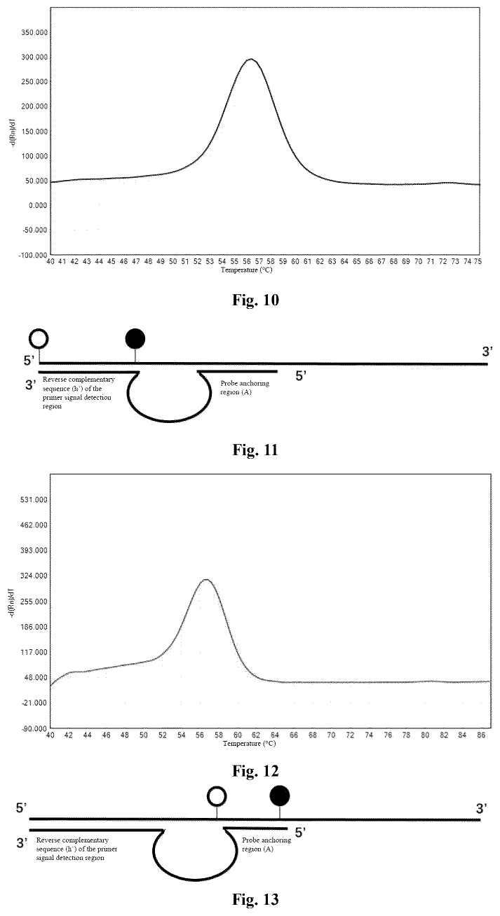

The detection results were subjected to data analysis by using an analysis software of the SLAN real-time fluorescence quantitative PCR instrument from Shanghai Hongshi Medical Technology Co., Ltd. The results were interpreted via a Tm value of the melting curve. FIG. 12 shows that a melting peak with a Tm value of 56.76° C. is formed in a PCR reaction well with addition of a non-small cell lung cancer EGFR exon 20 H773_V774insH mutation positive sample in an FAM channel

### Example 6: Singleplex Detection of Non-Small Cell Lung Cancer EGFR Exon 20 H773_V774insH Mutation by Using a Primer Probe System

TABLE 13
Primer probe

| Name of | | | |
|---|---|---|---|
| primer | | Nucleotide | |
| probe | Number | sequence (5′-3′) | Label |
| P7 | SEQ ID | GGCCGTAATCTGCACT | T20-CY5, |
| | NO: 25 | GCATCACATCGCCCGC | T33-BHQ2 |
| | | TAGCCCGTCCAATGCC | 3′Spacer C3 |
| R9 | SEQ ID | GGCCGTAATCTGCACT | / |
| | NO: 26 | AAAAGTCTGAACGACC | |
| | | TACACTCC | |
| F9 | SEQ ID | TAGCGGGCGATGTGAT | / |
| | NO: 27 | TTCCCAGCAGCTGACA | |
| | | CA | |

### in the Above Probe Primer Composition:

the forward primer F9 (SEQ ID NO: 27) is a specific primer designed for a target sequence of non-small cell lung cancer EGFR exon 20 H773_V774insH mutation, and the F9 has a total length of 34 bp, wherein a 1^{st} to 16^{th} bases at 3′ end thereof serve as a target sequence binding region, and a 1^{st} to 15^{th} bases at 5′ end thereof serve as a probe anchoring region (A), which is reverse complementary with a sequence of a 20^{th} to 34^{th} bases (i.e. a primer anchoring region (A′)) at 5′ end of the probe P7 (SEQ ID NO: 25); and
the reverse primer R9 (SEQ ID NO: 26) is a specific primer designed for the target sequence of non-small cell lung cancer EGFR exon 20 H773_V774insH mutation, and the R9 has a total length of 40 bp, wherein a 1^{st} to 21^{st} bases at 3′ end thereof serve as a target sequence binding region, a 1^{st} to 16^{th} bases at 5′ end thereof serve as a primer signal detection region (h), which is identical to a sequence of a 1^{st} to 16^{th} bases at 3′ end of the probe P7 (SEQ ID NO: 25)

A reaction system comprises the above primer probe, a template, a DNA polymerase, a reverse transcriptase, and a reaction buffer, and is used for target specific amplification and fluorescence signal detection. Specifically, the reaction system is involved in the following steps:
(1) Firstly, in a cycle of PCR amplification reaction, the forward primer (F) and the reverse primer (R) may specifically amplify a target nucleic acid sequence. After amplification, a double-stranded amplification product will be obtained, wherein one single-stranded amplification product (S1) comprises the probe anchoring region (A), the target sequence, and a reverse complementary sequence (h′) of the primer signal detection region from 5′ end to 3′ end; and the probe signal detection region (H) of the probe (P) is complementarily paired with the reverse complementary sequence (h′) of the primer signal detection region of the single-stranded amplification product (S1), and the primer anchoring region (A′) of the probe (P) is paired with the probe anchoring region (A) at 5′ end of the single-stranded amplification product (S1) in a reverse complementary manner. At this time, a distance between the first detectable label and the second detectable label of the probe (P) is changed, thereby generating a signal that may be detected by the instrument.
(2) After the PCR amplification is completed, a melting curve analysis is performed, and the double-stranded product formed by the probe (P) and the single-stranded amplification product (S1) will melt at a certain temperature, at this time, the probe (P) with the first detectable label and the second detectable label will be changed to the single-stranded state, thereby generating a fluorescence signal change that may be detected by the instrument.

The reaction system comprises the following components:

TABLE 14
Reaction system in this Example

| | |
|---|---|
| Reagents and components | Concentration |

| System for | 2× PCR Reaction Buffer | 1× | |
|---|---|---|---|
| singleplex | DNA Polymerase | 2 | U |
| detection of | Probe (P7) | 400 | nM |
| non-small | Forward primer (F9) | 500 | nM |
| cell lung | Reverse primer (R9) | 100 | nM |
| cancer EGFR | Nucleic acid template | 30 | copies |

| | | |
|---|---|---|
| exon 20 | Ultra-pure water | Adding to 20 μL |
| H773_V774insH | | |
| mutation | | |

A non-small cell lung cancer EGFR exon 20 H773_V774insH mutation was used as a positive sample, and the reverse primer (R) with a signal detection region sequence was used to separately detect the target sequence of non-small cell lung cancer EGFR exon 20 H773_V774insH mutation, to verify a detection ability for single mutation. The pure water was used as a no template control (NTC). A reaction principle was shown in FIG. 13.

The reaction buffer and a primer probe premixed solution were mixed according to the reaction system described in Table 14, a nucleic acid was extracted from a sample to be detected by a suitable method and was added into the prepared reaction system, and then a PCR amplification and a melting curve analysis were performed. An EGFR mutation type in the sample may be judged based on a characteristic Tm value of the target in a specific detection channel.

The detection was performed by using an SLAN real-time fluorescence quantitative PCR instrument and reagent consumables from Shanghai Hongshi Medical Technology Co., Ltd. The 2×PCR reaction buffer comprises 3 mM MgCl₂, 30 mM Tris-HCl with a pH of 8.3, 0.5 mM dNTP, 70 mM (NH₄)₂SO₄, etc.

### PCR Amplification and Melting Curve Analysis:

After a PCR tube was sealed by a cap, the sample was gently and uniformly mixed, and centrifuged for a short time and then left for 5 min at room temperature. The PCR tube was put in a handheld centrifuge again, centrifuged for a short time, and then transferred to a tray of the fluorescence quantitative PCR instrument. The procedure used was as follows: performing pre-denaturation at 95° C. for 3 minutes; performing denaturation at 94° C. for 10 seconds, and performing annealing and extension at 56° C. for 30 seconds, for a total of 45 cycles; and performing melting curve analysis at 40-85° C., with a heating rate of 0.05° C./s and with lighting.

### Data Analysis:

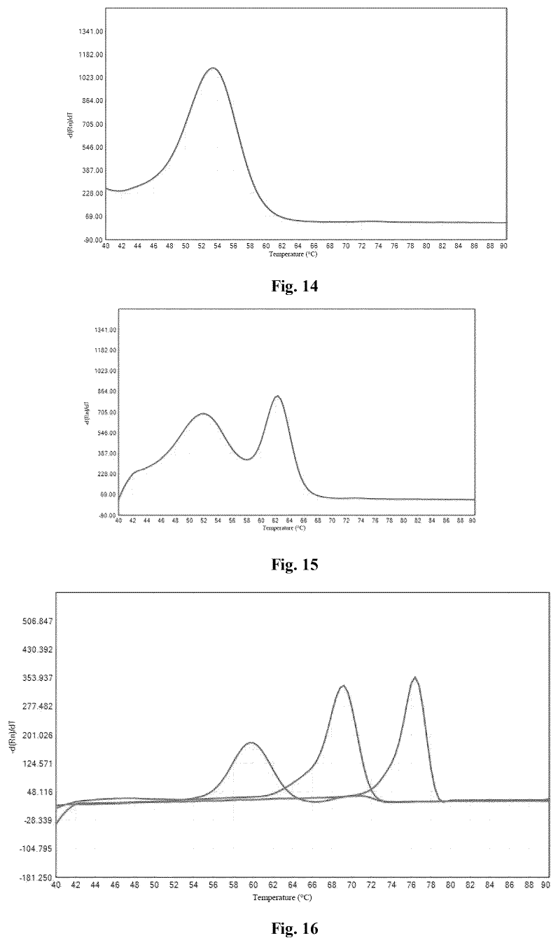

The detection results were subjected to data analysis by using an analysis software of the SLAN real-time fluorescence quantitative PCR instrument from Shanghai Hongshi Medical Technology Co., Ltd. The results were interpreted via a Tm value of the melting curve. FIG. 14 shows that a melting peak with a Tm value of 53.67° C. is formed by reverse complementation of the probe anchoring region (A) of the forward primer (F9) and the primer anchoring region (A′) of the probe (P7) in a PCR reaction well as negative control well (NTC) in a CY5 channel. FIG. 15 shows that a melting peak with a Tm value of 52.43° C. and a positive melting peak with a Tm value of 62.41° C. are formed by reverse complementation of the probe anchoring region (A) of the forward primer (F9) and the primer anchoring region (A′) of the probe (P7) in a PCR reaction well with addition of non small cell lung cancer EGFR exon 20 H773_V774insH mutation positive sample in a CY5 channel.

### Example 7: Multiplex PCR Detection of Non-Small Cell Lung Cancer EGFR Exon 20 T790M Mutation, Exon 21 L858R Mutation, and Exon 21 L861Q Mutation

TABLE 15
Primer probe

| Name of | | | |
|---|---|---|---|
| primer | | Nucleotide | |
| probe | Number | sequence (5′-3′) | Label |
| P8 | SEQ ID | AGACGCACGGTGACAT | T16-FAM, |
| | NO: 28 | GTGTTTGACTCAGTTC | T31-BHQ2 |
| | | TGCTGCTGCCCACCGT | 3′Spacer C3 |
| | | CTGGAGTGCGT | |
| F10 | SEQ ID | CACCGTGCGTCTAAAT | / |
| | NO: 29 | GATGGACAGCGTGGAC | |
| | | AA | |
| R10 | SEQ ID | TTTTTCATGTGTTTGA | / |
| | NO: 30 | CTCAGTTTTTCGAAGA | |
| | | GCATGAGCTGCA | |
| F11 | SEQ ID | CACCGTGCGTCTAAAG | / |
| | NO: 31 | TCAAGATCACAGATTT | |
| | | TGGGCG | |
| R11 | SEQ ID | CTGCTGCTGCTTTCTG | / |
| | NO: 32 | GCTGACCTAAAGCCAC | |
| | | C | |
| F12 | SEQ ID | CACCGTGCGTCTAAAA | / |
| | NO: 33 | GATTTTGGGCTGGCCA | |
| | | AACA | |
| R12 | SEQ ID | CTGGAGTGCGTTTTGC | / |
| | NO: 34 | CTCCTTCTGCATGGTA | |
| | | TTCT | |

### in the Above Probe Primer Composition:

Both of the forward primer F10 (SEQ ID NO: 29) and the reverse primer R10 (SEQ ID NO: 30) are specific primers designed for a target sequence of non-small cell lung cancer EGFR exon 20 T790M mutation. The F10 has a total length of 34 bp, wherein a 1^{st} to 19^{th} bases at 3′ end thereof serve as a target sequence binding region 1, and a 1^{st} to 12^{th} bases at 5′ end thereof serve as a probe anchoring region (A1), which is reverse complementary with a sequence of a 1^{st} to 12^{th} bases (i.e. a primer anchoring region (A1′)) of the probe P8 (SEQ ID NO: 28). The R10 has a total length of 44 bp, wherein a 1^{st} to 18^{th} bases at 3′ end thereof serve as a target sequence binding region 2, a 1^{st} to 5^{th} bases at 5′ end thereof serve as an extension blocking region, and a 6^{th} to 23^{rd} bases at 5′ end thereof serve as a primer signal detection region (h1), which is identical to a sequence of a 14^{th} to 31^{st} bases at 5′ end of the probe P8 (SEQ ID NO: 28).

Both of the forward primer F11 (SEQ ID NO: 31) and the reverse primer R11 (SEQ ID NO: 32) are specific primers designed for a target sequence of non-small cell lung cancer EGFR exon 21 L858R mutation. The F11 has a total length of 38 bp, wherein a 1^{st} to 23^{rd} bases at 3′ end thereof serve as a target sequence binding region 3, and a 1^{st} to 12^{th} bases at 5′ end thereof serve as a probe anchoring region (A1), which is reverse complementary with a sequence of a 1^{st} to 12^{th} bases (i.e. a primer anchoring region (A1′)) at 5′ end of the probe P8 (SEQ ID NO: 28). The R11 has a total length of 33 bp, wherein a 1^{st} to 20^{th} bases at 3′ end thereof serve as a target sequence binding region 4, a 1^{st} to 10^{th} bases at 5′ end thereof serve as a primer signal detection region (h2), which is identical to a sequence of a 19^{th} to 28^{th} bases at 3′ end of the probe P8 (SEQ ID NO: 28).

Both of the forward primer F12 (SEQ ID NO: 33) and the reverse primer R12 (SEQ ID NO: 34) are specific primers designed for a target sequence of non-small cell lung cancer EGFR exon 21 L861Q mutation. The F12 has a total length of 36 bp, wherein a 1^{st} to 21^{st} bases at 3′ end thereof serve as a target sequence binding region 5, and a 1^{st} to 12^{th} bases at 5′ end thereof serve as a probe anchoring region (A1), which is reverse complementary with a sequence of a 1^{st} to 12^{th} bases (i.e. a primer anchoring region (A1′)) at 5′ end of the probe P8 (SEQ ID NO: 28). The R12 has a total length of 36 bp, wherein a 1^{st} to 22^{nd} bases at 3′ end thereof serve as a target sequence binding region 6, a 1^{st} to 10^{th} bases at 5′ end thereof serve as a primer signal detection region (h3), which is identical to a sequence of a 1^{st} to 10^{th} bases at 3′ end of the probe P8 (SEQ ID NO: 28).

The characteristic Tm values of the above probes are shown in Table 16:

TABLE 16

| Fluorescent | Melting temperature (Tm, ° C.) | | |
|---|---|---|---|
| channel | 56 ≤ Tm < 62 | 64 ≤ Tm < 72 | 72 ≤ Tm < 80 |
| FAM | T790M mutation | L858R mutation | L861Q mutation |

A multiplex PCR detection for non small cell lung cancer EGFR exon 20 T790M mutation, exon 21 L858R mutation, and exon 21 L861Q mutation was performed by using the above primer probe.

A reaction system used for detection was shown in Table 17.

TABLE 17
Reaction system for multiplex PCR detection of non
small cell lung cancer EGFR exon 20 T790M mutation,
exon 21 L858R mutation, and exon 21 L861Q mutation

| Reagents and components | Concentration | |
|---|---|---|
| 2× PCR Reaction Buffer | 1× | |
| DNA Polymerase | 2 | U |
| Probe (P8) | 400 | nM |
| Forward primer (F10) | 500 | nM |
| Reverse primer (R10) | 100 | nM |
| Forward primer (F11) | 500 | nM |
| Reverse primer (R11) | 100 | nM |
| Forward primer (F12) | 500 | nM |
| Reverse primer (R12) | 100 | nM |
| Non small cell lung cancer EGFR exon | 400 | copies |

| | |
|---|---|
| 20 T790M mutation nucleic acid sample | |
| or exon 21 L858R mutation nucleic acid | |
| sample or exon 21 L861Q mutation | |
| nucleic acid sample | |
| Ultra-pure water | Adding to 20 μL |

Note: The 2×PCR reaction buffer comprises 3 mM MgCl₂, 30 mM Tris-HCl buffer with a pH of 8.3, 0.5 mM dNTP, and 70 mM (NH₄)₂SO₄.

Nucleic acid template: a non small cell lung cancer EGFR exon 20 T790M mutation nucleic acid sample or exon 21 L858R mutation nucleic acid sample or exon 21 L861Q mutation nucleic acid sample was used as the nucleic acid template respectively, to verify a detection ability for single gene mutation in a multiplex system. The pure water was used as a no template control (NTC).

### Reaction Preparation:

After the preparation of the nucleic acid templates was completed, the reaction system was prepared according to the ratios described in Table 17.

### PCR Amplification and Melting Curve Analysis:

After a PCR tube was sealed by a cap, the sample was gently and uniformly mixed, and centrifuged for a short time and then left for 5 min at room temperature. The PCR tube was put in a handheld centrifuge again, centrifuged for a short time, and then transferred to a tray of SLAN real-time fluorescence quantitative PCR instrument from Shanghai Hongshi Medical Technology Co., Ltd. The procedure used was as follows: performing pre-denaturation at 95° C. for 3 minutes; performing denaturation at 94° C. for 10 seconds, and performing annealing and extension at 56° C. for 30 seconds, for a total of 45 cycles, without lighting; performing denaturation at 94° C. for 150 seconds, and performing incubation at a constant temperature of 40° C. for 300 seconds; and performing melting curve analysis at 62-86° C., with a heating rate of 0.05° C./s and with lighting, to obtain melting curves.

### Data Analysis:

The results were interpreted via Tm values of the melting curves by using an analysis software of the SLAN real-time fluorescence quantitative PCR instrument from Shanghai Hongshi Medical Technology Co., Ltd.

FIG. 16 shows that melting peaks of non-small cell lung cancer EGFR exon 20 T790M mutation (59.81° C.), non-small cell lung cancer EGFR exon 21 L858R mutation (69.13° C.), and non-small cell lung cancer EGFR exon 21 L861Q mutation (76.31° C.) appear in PCR reaction wells with addition of non small cell lung cancer EGFR exon 20 T790M mutation nucleic acid sample, exon 21 L858R mutation nucleic acid sample and exon 21 L861Q mutation nucleic acid sample in an FAM channel, respectively.

1. A probe primer composition for PCR detection, comprising a probe and a first primer set, wherein the first primer set comprises a first primer and a second primer that are a forward primer and a reverse primer or a reverse primer and a forward primer, respectively,
the probe comprises a first binding region A′ and a second binding region H,
the first primer comprises a sequence region A that is partially or completely complementary with the first binding region A′ of the probe, and the second primer comprises a sequence region h that is partially or completely identical to the second binding region H of the probe,
none of the first binding region A′, the second binding region H, the sequence region A and the sequence region h are complementary or identical to a target sequence or any fragment thereof, and
the probe has a detectable label thereon.

2. The probe primer composition of claim 1, wherein the first primer further comprises a first target sequence binding region, and the second primer further comprises a second target sequence binding region.

3. The probe primer composition of claim 1, wherein the first primer further comprises an amplification product capturing region B, and the probe primer composition further comprises a second primer set, the second primer set comprises a third primer and the second primer that are a forward primer and a reverse primer or a reverse primer and a forward primer, respectively, the third primer comprises a third target sequence binding region and an amplification product capturing region B, and the second primer further comprises a second target sequence binding region.

4. The probe primer composition of claim 1, wherein the second primer further comprises an amplification product capturing region C, and the probe primer composition further comprises a second primer set, the second primer set comprises the first primer and a fourth primer that are a forward primer and a reverse primer or a reverse primer and a forward primer, respectively, wherein the fourth primer comprises a fourth target sequence binding region and an amplification product capturing region C, and the first primer further comprises a first target sequence binding region.

5. The probe primer composition of claim 1, wherein the first primer further comprises an amplification product capturing region B, the second primer further comprises an amplification product capturing region C, and the probe primer composition further comprises a second primer set, the second primer set comprises a third primer and a fourth primer that are a forward primer and a reverse primer or a reverse primer and a forward primer, respectively, the third primer set comprises a third target sequence binding region and an amplification product capturing region B, and the fourth primer comprises a fourth target sequence binding region and an amplification product capturing region C.

6. The probe primer composition of claim 1, wherein a single-stranded amplification product of the first primer and the second primer comprises the sequence region A and a sequence h′ that is partially or completely complementary with the second binding region H of the probe.

7. The probe primer composition of claim 1, the first binding region A′ is located at a 5′ end or 3′ end of the probe, and preferably, a blocking region is provided at the 3′ end of the probe.

8. The probe primer composition of claim 1, wherein the detectable label comprises a first detectable label and a second detectable label, the first detectable label is a fluorescence reporter, and the second detectable label is a quencher or other labeling groups that can produce a signal change with the first detectable label through fluorescence resonance energy transfer,
preferably, both of the first detectable label and the second detectable label are located on the first binding region A′ or both of the first detectable label and the second detectable label are located on the second binding region H; or
preferably, a spacer region between the first detectable label and the second detectable label is at least partially located on the first binding region A′, or the spacer region between the first detectable label and the second detectable label is at least partially located on the second binding region H.

9. The probe primer composition of claim 2, wherein the first primer sequentially comprises the sequence region A and the first target sequence binding region from 5′ end to 3′ end;
the second primer sequentially comprises the sequence region h and the second target sequence binding region from 5′ end to 3′ end,
preferably, the second primer further comprises an extension blocking region, which is a sequence that is not identical to or complementary with a base sequence of any part of the probe, and
more preferably, the second primer sequentially comprises the extension blocking region, the sequence region h, and the second target sequence binding region from 5′ end to 3′ end.

10. The probe primer composition of claim 2, wherein the probe primer composition for n target sequences comprises n first primer sets and m probes, wherein 1≤n≤20, 1≤m≤n;
preferably, the probe primer composition for n target sequences comprises n first primer sets and 1 or several probes, wherein 1≤n≤20;
preferably, single-stranded amplification products obtained by amplifying different target sequences with all of the first primer sets are different from each other in an annealing temperature with the probe,
preferably, single-stranded amplification products obtained by amplifying different target sequences with all of the first primer sets are different from each other by a ΔTm of 6-10° C. in an annealing temperature with the probe, and
preferably, the target sequence binding regions included in all of the first primers are different from each other, and the target sequence binding regions included in all of the second primers are different from each other.

11. The probe primer composition of claim 3, wherein the third primer sequentially comprises the amplification product capturing region B and the third target sequence binding region from 5′ end to 3′ end; the first primer sequentially comprises the sequence region A and the amplification product capturing region B from 5′ end to 3′ end; and the second primer sequentially comprises the sequence region h and the second target sequence binding region from 5′ end to 3′ end;
the amplification product capturing region B does not bind with the target sequence or any fragment thereof in a paired manner;
preferably, the second primer further comprises an extension blocking region, which is a sequence that is not identical to or complementary with a base sequence of any part of the probe; and
more preferably, the second primer sequentially comprises the extension blocking region, the sequence region h, and the second target sequence binding region from 5′ end to 3′ end.

12. The probe primer composition of claim 3, wherein the probe primer composition for n target sequences comprises n second primer sets, m probes, and o first primers, wherein 1≤n≤20, 1≤m≤n, and 1≤o≤n,
preferably, the probe primer composition for n target sequences comprises n second primer sets, 1 or several probes, and 1 or several first primers, wherein 1≤n≤20,
preferably, single-stranded amplification products obtained by amplifying with all of the first primer sets are different from each other in an annealing temperature with the probe,
preferably, single-stranded amplification products obtained by amplifying with all of the first primer sets are different from each other by a ΔTm of 6-10° C. in an annealing temperature with the probe, and
preferably, the target sequence binding regions included in all of the third primers are different from each other, and the target sequence binding regions included in all of the second primers are different from each other.

13. The probe primer composition of claim 4, wherein the first primer sequentially comprises the sequence region A and the first target sequence binding region from 5′ end to 3′ end; the second primer sequentially comprises the sequence region h and the amplification product capturing region C from 5′ end to 3′ end; and the fourth primer sequentially comprises the amplification product capturing region C and the fourth target sequence binding region from 5′ end to 3′ end;
the amplification product capturing region C does not bind with the target sequence or any fragment thereof in a paired manner;
preferably, the second primer further comprises an extension blocking region, which is a sequence that is not identical to or complementary with a base sequence of any part of the probe; and
more preferably, the second primer sequentially comprises the extension blocking region, the sequence region h, and the amplification product capturing region C from 5′ end to 3′ end.

14. The probe primer composition of claim 4, wherein the probe primer composition for n target sequences comprises n second primer sets, m probes, and o second primers, wherein 1≤n≤20, 1≤m≤n, and 1≤o≤n,
preferably, the probe primer composition for n target sequences comprises n second primer sets, 1 or several probes, and 1 or several second primers, wherein 1≤n≤20,
preferably, single-stranded amplification products obtained by amplifying with all of the first primer sets are different from each other in an annealing temperature with the probe,
preferably, single-stranded amplification products obtained by amplifying with all of the first primer sets are different from each other by a ΔTm of 6-10° C. in an annealing temperature with the probe, and
preferably, the target sequence binding regions included in all of the first primers are different from each other, and the target sequence binding regions included in all of the fourth primers are different from each other.

15. The probe primer composition of claim 5, wherein the first primer sequentially comprises the sequence region A and the amplification product capturing region B from 5′ end to 3′ end; the second primer sequentially comprises the sequence region h and the amplification product capturing region C from 5′ end to 3′ end; the third primer sequentially comprises the amplification product capturing region B and the third target sequence binding region from 5′ end to 3′ end; and the fourth primer sequentially comprises the amplification product capturing region C and the fourth target sequence binding region from 5′ end to 3′ end;
neither the amplification product capturing region B nor the amplification product capturing region C is paired with the target sequence or any fragment thereof;
preferably, the second primer further comprises an extension blocking region, which is a base sequence that is not identical to or complementary with the probe or any fragment thereof, and
more preferably, the second primer sequentially comprises the extension blocking region, the sequence region h, and the amplification product capturing region C from 5′ end to 3′ end.

16. The probe primer composition of claim 5, wherein the probe primer composition for n target sequences comprises n second primer sets, m probes, and p first primer sets, wherein 1≤n≤20, 1≤m≤n, and 1≤p≤n,
preferably, the probe primer composition for n target sequences comprises n second primer sets, 1 or several probes, and 1 or several first primer sets, wherein 1≤n≤20,
preferably, single-stranded amplification products obtained by amplifying with all of the first primer sets are different from each other in an annealing temperature with the probe,
preferably, single-stranded amplification products obtained by amplifying with all of the first primer sets are different from each other by a ΔTm of 6-10° C. in an annealing temperature with the probe, and
preferably, the target sequence binding regions included in all of the third primers are different from each other, and the target sequence binding regions included in all of the fourth primers are different from each other.

17. (canceled)

18. A kit for PCR detection, comprising the probe primer composition of claim 1.

19. A method for PCR detection, comprising steps of performing PCR by using the probe primer composition of claim 1, comprising:
using the first primer set to specifically bind with a target sequence and extend to produce a double-stranded pre-amplification product, wherein one single-stranded amplification product of the double-stranded pre-amplification product comprises the sequence region A, the target sequence, and a complementary sequence h′ of the sequence region h; and
forming a signal change, by specifical binding of the sequence region A and the complementary sequence h′ of the single-stranded amplification product with the probe, that can be detected by an instrument, and determining whether the target sequence is present based on the signal change;
preferably, the single-stranded amplification product further comprises a complementary sequence of the extension blocking region at 3′ end thereof, which is not complementary with any part of the probe.

20. (canceled)

21. The method of claim 19, the method comprises the following steps:
using the third primer and the second primer of the second primer set to specifically bind with a target sequence and extend to produce a first double-stranded pre-amplification product, wherein one single-stranded amplification product of the first double-stranded pre-amplification product comprises a complementary sequence B′ of the amplification product capturing region B, the target sequence, and the sequence region h;
using the first primer set and taking the first double-stranded pre-amplification product as a template, to extend to obtain a second double-stranded amplification product, wherein one single-stranded amplification product of the second double-stranded amplification product comprises the sequence region A, the amplification product capturing region B, the target sequence, and a complementary sequence h′ of the sequence region h;
forming a signal change, by specifical binding of the sequence region A and the complementary sequence h′ of the single-stranded amplification product with the probe, that can be detected by an instrument, and determining whether the target sequence is present based on the signal change;
preferably, the one single-stranded amplification product of the first double-stranded pre-amplification product further comprises an extension blocking region, and/or the one single-stranded amplification product of the second double-stranded amplification product further comprises a complementary sequence of the extension blocking region, and the complementary sequence of the extension blocking region is not complementary with any part of the probe.

22. The method of claim 19, comprising the following steps:
using the first primer and the fourth primer of the second primer set to specifically bind with a target sequence and extend to produce a first double-stranded pre-amplification product, wherein one single-stranded amplification product of the first double-stranded pre-amplification product comprises a complementary sequence A′ of the sequence region A, the target sequence, and the amplification product capture region C;
using the first primer set and taking the first double-stranded pre-amplification product as a template to extend to obtain a second double-stranded amplification product, wherein one single-stranded amplification product of the second double-stranded amplification product comprises the sequence region A, the target sequence, a complementary sequence C′ of the amplification product capturing region C, and a complementary sequence h′ of the sequence region h, and
forming a signal change, by specifical binding of the sequence region A and the complementary sequence h′ of the single-stranded amplification product with the probe, that can be detected by an instrument, and determining whether the target sequence is present based on the signal change;
preferably, the one single-stranded amplification product of the second double-stranded amplification product further comprises a complementary sequence of the extension blocking region, and the complementary sequence of the extension blocking region is not complementary with any part of the probe.

23. The method of claim 19, comprising the following steps:
using the third primer and the fourth primer of the second primer set to specifically bind with a target sequence and extend to produce a first double-stranded pre-amplification product, wherein one single-stranded amplification product of the first double-stranded pre-amplification product comprises a complementary sequence B′ of the amplification product capturing region B, the target sequence, and the amplification product capture region C;
using the first primer set and taking the first double-stranded pre-amplification product as a template to extend to obtain a second double-stranded amplification product, wherein one single-stranded amplification product of the second double-stranded amplification product comprises the sequence region A, the amplification product capturing region B, the target sequence, a complementary sequence C′ of the amplification product capturing region C, and a complementary sequence h′ of the sequence region h, and
forming a signal change, by specifical binding of the sequence region A and the complementary sequence h′ of the single-stranded amplification product with the probe, that can be detected by an instrument, and determining whether the target sequence is present based on the signal change; wherein,
preferably, the one single-stranded amplification product of the second double-stranded amplification product further comprises a complementary sequence of the extension blocking region, and the complementary sequence of the extension blocking region is not complementary with any part of the probe.

24. The method of claim 19, wherein the signal change refers to a fluorescence signal change or a change in melting temperature curve; and preferably, the signal change refers to the change in melting temperature curve.

25.-27. (canceled)


SEQUENCE LISTING (34 sequences)

SEQ ID NO: 1 - DNA, 40 bp, synthetic construct
  source 1..40: mol_type = other DNA
  modified_base 40: mod_base = OTHER; note = C3 (three carbon) spacer conjugated to 3' end of the deoxyribose sugar
  modified_base 14: mod_base = OTHER; note = CY5 (Cyanine 5) conjugated to T (thymine) base
  modified_base 26: mod_base = OTHER; note = BHQ1 (Black Hole Quencher 1) conjugated to T (thymine) base
ccctgggagctcatggaggtactggtcctgtcgacgcacg

SEQ ID NO: 2 - DNA, 55 bp, synthetic construct
  source 1..55: mol_type = other DNA
ttatttcatggaggtactggtcctgtcgaaatattgcagatccwacacctaacaa

SEQ ID NO: 3 - DNA, 42 bp, synthetic construct
  source 1..42: mol_type = other DNA
gctcccagggtttaacattaagcaagaagaggaaamgaagat

SEQ ID NO: 4 - DNA, 41 bp, synthetic construct
  source 1..41: mol_type = other DNA
  modified_base 16: mod_base = OTHER; note = VIC (Victoria) conjugated to T (thymine) base
  modified_base 27: mod_base = OTHER; note = BHQ2 (Black Hole Quencher 2) conjugated to T (thymine) base
  modified_base 41: mod_base = OTHER; note = C3 (three carbon) spacer conjugated to 3' end of the deoxyribose sugar
cgcaagtgaggcgactacgtgagcgatggacggaggcagga

SEQ ID NO: 5 - DNA, 37 bp, synthetic construct
  source 1..37: mol_type = other DNA
gcctcacttgcgaaaaagcaaccaacaacattcattg

SEQ ID NO: 6 - DNA, 42 bp, synthetic construct
  source 1..42: mol_type = other DNA
tttttctacgtgagcgatggaaagcaattcagcatcacagac

SEQ ID NO: 7 - DNA, 35 bp, synthetic construct
  source 1..35: mol_type = other DNA
gcctcacttgcgaaaataatcaggcacgagaacag

SEQ ID NO: 8 - DNA, 52 bp, synthetic construct
  source 1..52: mol_type = other DNA
tttttgactacgtgagcgatggacggaggaaaagactagcaacctccatggc

SEQ ID NO: 9 - DNA, 35 bp, synthetic construct
  source 1..35: mol_type = other DNA
  modified_base 14: mod_base = OTHER; note = FAM (fluorescein amidite) conjugated to T (thymine) base
  modified_base 26: mod_base = OTHER; note = BHQ1 (Black Hole Quencher 1) conjugated to T (thymine) base
  modified_base 35: mod_base = OTHER; note = C3 (three carbon) spacer conjugated to 3' end of the deoxyribose sugar
ccctgggagctcatggaggtactggtcctgtcgac

SEQ ID NO: 10 - DNA, 20 bp, synthetic construct
  source 1..20: mol_type = other DNA
  modified_base 20: mod_base = OTHER; note = BHQ1 (Black Hole Quencher 1) conjugated to T (thymine) base
  modified_base 1: mod_base = OTHER; note = FAM (fluorescein amidite) conjugated to A (adenine) base
agcgacggtgccgagagggt

SEQ ID NO: 11 - DNA, 55 bp, synthetic construct
  source 1..55: mol_type = other DNA
ttatttcatggaggtactggtcctgtcgaaatattgcagatactccacctcatca

SEQ ID NO: 12 - DNA, 42 bp, synthetic construct
  source 1..42: mol_type = other DNA
gctcccagggtttaacaggctgcaagaagaggactagaagat

SEQ ID NO: 13 - DNA, 47 bp, synthetic construct
  source 1..47: mol_type = other DNA
agcgacggtgccgagagggtaaatattgcagatactccacctcatca

SEQ ID NO: 14 - DNA, 29 bp, synthetic construct
  source 1..29: mol_type = other DNA
aacaggctgcaagaagaggactagaagat

SEQ ID NO: 15 - DNA, 33 bp, synthetic construct
  source 1..33: mol_type = other DNA
ttttttttttgatgaggtggagtatctgcaata

SEQ ID NO: 16 - DNA, 48 bp, synthetic construct
  source 1..48: mol_type = other DNA
ttatttggaggtactggtcctaaatattgcagatactccacctcatca

SEQ ID NO: 17 - DNA, 44 bp, synthetic construct
  source 1..44: mol_type = other DNA
gactgcagtcaatttaacaggctgcaagaagaggactagaagat

SEQ ID NO: 18 - DNA, 25 bp, synthetic construct
  source 1..25: mol_type = other DNA
gctcccagggaaagactgcagtcaa

SEQ ID NO: 19 - DNA, 40 bp, synthetic construct
  source 1..40: mol_type = other DNA
cgcattacaagttaaatattgcagatactccacctcatca

SEQ ID NO: 20 - DNA, 37 bp, synthetic construct
  source 1..37: mol_type = other DNA
ttatttggaggtactggtcctaaacgcattacaagtt

SEQ ID NO: 21 - DNA, 35 bp, synthetic construct; identical to SEQ ID NO: 9
  source 1..35: mol_type = other DNA
  modified_base 14: mod_base = OTHER; note = CY5 (Cyanine 5) conjugated to T (thymine) base
  modified_base 26: mod_base = OTHER; note = BHQ2 (Black Hole Quencher 2) conjugated to T (thymine) base
  modified_base 35: mod_base = OTHER; note = C3 (three carbon) spacer conjugated to 3' end of the deoxyribose sugar

SEQ ID NO: 22 - DNA, 44 bp, synthetic construct
  source 1..44: mol_type = other DNA
  modified_base 1: mod_base = OTHER; note = FAM (fluorescein amidite) conjugated to C (cytosine) base
  modified_base 15: mod_base = OTHER; note = BHQ1 (Black Hole Quencher 1) conjugated to T (thymine) base
  modified_base 44: mod_base = OTHER; note = C3 (three carbon) spacer conjugated to 3' end of the deoxyribose sugar
ccacgtctcaactatgacactcctccgaccatccgcttcctcca

SEQ ID NO: 23 - DNA, 39 bp, synthetic construct
  source 1..39: mol_type = other DNA
ccacgtctcaactataaaagtctgaacgacctacactcc

SEQ ID NO: 24 - DNA, 34 bp, synthetic construct
  source 1..34: mol_type = other DNA
tggaggaagcggatgtttcccagcagctgacaca

SEQ ID NO: 25 - DNA, 48 bp, synthetic construct
  source 1..48: mol_type = other DNA
  modified_base 20: mod_base = OTHER; note = CY5 (Cyanine 5) conjugated to T (thymine) base
  modified_base 33: mod_base = OTHER; note = BHQ2 (Black Hole Quencher 2) conjugated to T (thymine) base
  modified_base 48: mod_base = OTHER; note = C3 (three carbon) spacer conjugated to 3' end of the deoxyribose sugar
ggccgtaatctgcactgcatcacatcgcccgctagcccgtccaatgcc

SEQ ID NO: 26 - DNA, 40 bp, synthetic construct
  source 1..40: mol_type = other DNA
ggccgtaatctgcactaaaagtctgaacgacctacactcc

SEQ ID NO: 27 - DNA, 34 bp, synthetic construct
  source 1..34: mol_type = other DNA
tagcgggcgatgtgatttcccagcagctgacaca

SEQ ID NO: 28 - DNA, 59 bp, synthetic construct
  source 1..59: mol_type = other DNA
  modified_base 16: mod_base = OTHER; note = FAM (fluorescein amidate) conjugated to T (thymine) base
  modified_base 31: mod_base = OTHER; note = BHQ2 (Black Hole Quencher 2) conjugated to T (thymine) base
  modified_base 59: mod_base = OTHER; note = C3 (three carbon) spacer conjugated to 3' end of the deoxyribose sugar
agacgcacggtgacatgtgtttgactcagttctgctgctgcccaccgtctggagtgcgt

SEQ ID NO: 29 - DNA, 34 bp, synthetic construct
  source 1..34: mol_type = other DNA
caccgtgcgtctaaatgatggacagcgtggacaa

SEQ ID NO: 30 - DNA, 44 bp, synthetic construct
  source 1..44: mol_type = other DNA
tttttcatgtgtttgactcagtttttcgaagagcatgagctgca

SEQ ID NO: 31 - DNA, 38 bp, synthetic construct
  source 1..38: mol_type = other DNA
caccgtgcgtctaaagtcaagatcacagattttgggcg

SEQ ID NO: 32 - DNA, 33 bp, synthetic construct
  source 1..33: mol_type = other DNA
ctgctgctgctttctggctgacctaaagccacc

SEQ ID NO: 33 - DNA, 36 bp, synthetic construct
  source 1..36: mol_type = other DNA
caccgtgcgtctaaaagattttgggctggccaaaca

SEQ ID NO: 34 - DNA, 36 bp, synthetic construct
  source 1..36: mol_type = other DNA
ctggagtgcgttttgcctccttctgcatggtattct